(19) United States
(12) Patent Application Publication
(10) Pub. No.: US 2007/0123556 A1
(43) Pub. Date: May 31, 2007

(54) Treatment with Sigma Receptor Agonists Post-Stroke

(75) Inventors: Keith R. Pennypacker, Wesley Chapel, FL (US); Javier Cuevas, Lutz, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 11/422,505

(22) Filed: Jun. 6, 2006

Related U.S. Application Data
(60) Provisional application No. 60/687,700, filed on Jun. 6, 2005.

(51) Int. Cl. A61K 31/473; A61K 31/195; A61K 31/155

(52) U.S. Cl. 514/289; 514/634; 514/561

(57) ABSTRACT

A method of post-stroke treatment at delayed timepoints with sigma receptor agonists. Sigma receptors are promising targets for neuroprotection following ischemia. One of the key components in the demise of neurons following ischemic injury is the disruption of intracellular calcium homeostasis. The sigma receptor agonist, DTG, was shown to depress [Ca²⁺]ᵢ elevations observed in response to ischemia induced by sodium azide and glucose deprivation. Two sigma receptor antagonists, metaphit and BD-1047, were shown to blunt the ability of DTG to inhibit ischemia-evoked increases in [Ca²⁺]ᵢ. DTG inhibition of ischemia-induced increases in [Ca²⁺]ᵢ was mimicked by the sigma-1 receptor-selective agonists, carbetapentane, (+)-pentazocine and PRE-084, but not by the sigma-2 selective agonist, ibogaine, showing that activation of sigma-1 receptors is responsible for the effects. Activation of sigma receptors can ameliorate [Ca²⁺]ᵢ dysregulation associated with ischemia in cortical neurons, providing neuroprotective properties. The effects of 1,3-di-o-tolyguanidine (DTG), a high affinity sigma receptor agonist, as a potential treatment for decreasing infarct area at delayed time points was further examined in rats. DTG treatment significantly reduced infarct area in both cortical/striatal and cortical/hippocampal regions by >80%, relative to control rats. These findings were confirmed by immunohistochemical experiments using the neuronal marker, mouse anti-neuronal nuclei monoclonal antibody (NeuN), which showed that application of DTG significantly increased the number of viable neurons in these regions. Furthermore, DTG blocked the inflammatory response evoked by MCAO, as indicated by decreases in the number of reactive astrocytes and activated microglia/macrophages detected by immunostaining for glial fibrillary acidic protein (GFAP) and binding of isolectin IB4, respectively. Thus, the sigma receptor-selective agonist, DTG, can enhance neuronal survival when administered 24 hr after an ischemic stroke. In addition, the efficacy of sigma receptors for stroke treatment at delayed time points is likely the result of combined neuroprotective and anti-inflammatory properties of these receptors.

### CROSS REFERENCE TO RELATED APPLICATIONS

This application claims priority to currently pending U.S. Provisional Patent Application 60/687,700, entitled, “Effective Treatment with Sigma Receptor Agonists Post-Stroke”, filed Jun. 6, 2005, the contents of which are herein incorporated by reference.

### STATEMENT OF GOVERNMENT INTEREST

This invention was made with Government support under Grant No. NIH NS39141 awarded by the National Institute of Health. Additional support has been provided by the American Heart Association under grant numbers AHA 0255016B and 0455210B. The Government has certain rights in the invention.

### FIELD OF INVENTION

This invention relates to the treatment of stroke. More specifically, this invention relates to stroke treatment at delayed time-points with sigma receptor agonists.

### BACKGROUND OF THE INVENTION

Stroke is the leading cause of severe disability and the third leading cause of death in the United States of America (AHA 2005). Intravenous application of recombinant tissue plasminogen activator (tPA), a thrombolytic agent, is the only FDA approved treatment for stroke and has a very limited therapeutic time window (New England Journal of Medicine 1995). This “clot-buster” must be administered within three hours of stroke onset (Albers et al., 2004), and can produce possible adverse effects such as hemorrhage and reperfusion damage from oxygen free radicals (Hacke et al., 1999; Kumura et al., 1996; Peters et al., 1998). The limitations and adverse effects of tPA have stimulated the search for alternative treatments for stroke.

When a cerebral embolic stroke occurs, a thrombus blocks blood perfusion to the brain and triggers a series of events that ultimately result in neuronal death. The disruption in blood supply directly results in the cessation of oxygen and nutrient delivery, which metabolically compromises the neurons and produces an infarction.

The infarct zone contains two regions associated with ischemic cell death. The center of the infarction or “core” is the area directly affected by the decrease in blood perfusion, and is where the greatest concentration of cell death can be found.

Surrounding the core is the penumbra, a region with diminished blood flow but where collaterals provide some oxygen and nutrients. However, perfusion in the penumbra is sufficiently reduced resulting in arrested physiological function and some degeneration of neurons (Ginsberg, 2003).

Neuronal death is enhanced by secondary inflammation caused by the immune response in the penumbra. The inflammatory response is primarily from resident activated microglia and infiltrating macrophages, which enter the central nervous system through the degrading blood brain barrier (Stoll et al., 1998). Reactive astrocytes and microglia exacerbate cerebral inflammation via their production of pro-inflammatory cytokines and chemokines (Trendelenburg and Dirnagl, 2005). These immune cells, which normally protect the brain via destruction of pathogens and promotion of tissue repair, become overactivated, and further promote the expansion of tissue damage by releasing high levels of nitric oxide (NO), glutamate, tumor necrosis factor-α (TNF-α), and interleukin-1 (IL-1)(Bal-Price and Brown, 2001; Heales et al., 1999; Hertz et al., 2001).

Therapeutic interventions that provide neuroprotection and reduce inflammation to decrease damage to the penumbra, thus reducing the extent of the damage produced by stroke, offer great promise for treatment. Recent studies showing that transfusion with human umbilical cord blood cells (HUCBC), which possess both of these properties, results in both infarct volume reduction and expansion of the therapeutic window, lend support to this conclusion (Newcomb et al., 2005; Vendrame M et al., 2005; Vendrame et al., 2004). Activation of sigma receptors has been shown to decrease neuronal death associated with hypoxia and to elicit an anti-inflammatory response (Bourrie et al., 2002; Goyagi et al., 2001). Thus, activation of sigma receptors may mimic the effects of HUCBC following stroke and provide for the expansion of the therapeutic time window for treatment after such injury.

Sigma receptors are widely distributed in the mammalian brain; and these receptors recognize a diverse array of centrally acting substances including opiates, antipsychotics, antidepressants, phencyclidine (PCP)-related compounds, and neurosteroids (Walker et al., 1990; Bowen, 2000). Thus far, two sigma receptor subtypes have been identified on the basis of their pharmacological profile, with the sigma-I receptor showing high affinity for positive isomer of bezomorphas such as (+)-pentazocine and (+)-SKF-10,047, and the sigma-2 receptor having high affinity for ibogaine (Vilner and Bowen, 2000), but only the sigma-1 receptor has been cloned (Hanner et al., 1996). While the function of sigma receptors is not well understood, they have been implicated in numerous physiological and pathophysiological processes such as learning and memory (Senda et al., 1996; Hiramatsu et al., 2006), movement disorders (Matsumoto et al., 1990), and drug addiction (McCracken et al., 1999). Sigma receptors are emerging as therapeutic targets for various diseases such neuropsychiatric disorders and cancer (Casellas et al., 2004; Hayashi and Su, 2004). Moreover, the observation that several sigma receptor ligands are neuroprotective in both in vivo and in vitro models of ischemia has generated interest in targeting these receptors to enhance neuronal survival following stroke (Takahashi et al., 1996; Lockhart et al., 1995).

Dysregulation of intracellular calcium homeostasis greatly contributes to the demise of neurons following an ischemic insult in the central nervous system (Mattson, 2000). Elevation of intracellular calcium disrupts plasma membrane function via activation of calcium-sensitive ion channels (Murai et al., 1997), and triggers biochemical cascades that ultimately promote processes such as proteolysis, lipolysis, and the production of reactive oxygen species (Mattson, 2000). It has been suggested that the neuroprotective properties of sigma ligands depend in part on their ability to depress elevations in intracellular calcium associated with glutamate receptor-mediated excitotoxicity (Klette et al., 1995; Klette et al., 1997). However, the membrane dysfunction produced by ischemia can stimulate multiple plasma membrane calcium fluxes, including Ca²⁺ fluxes attributable to voltage-gated calcium channels and independent of glutamate receptor activation (Nikonenko et al., 2005, Tanaka et al., 1997). Sigma receptors have been shown to block both voltage-gated calcium channels and ionotropic glutamate receptors (Zhang and Cuevas, 2002; Monnet et al., 2003). Both of these ion channels are believed to be involved in the dysregulation of intracellular calcium homeostasis accompanying ischemia, and selective inhibitors of these channels have been shown to provide some degree of neuroprotection (Schurr, 2004). Thus, one of the mechanisms by which sigma receptors may prevent these increases in calcium is via the inhibition of multiple plasma membrane calcium channels. However, the role of sigma receptors in the modulation of ischemia-induced elevations in intracellular calcium has not been unequivocally established because studies on the effects of sigma receptors on calcium homeostasis during neuronal injury have examined intracellular calcium changes in response to direct glutamate application rather than in vitro ischemia models. Given non-glutamate induced calcium fluxes are also a factor following ischemia, it is important to examine sigma receptor modulation of intracellular calcium using an ischemia model. Determining the role of sigma receptors in preserving calcium homeostasis is the first step toward establishing these receptors as a target for neuroprotection.

The studies of sigma receptor modulation of glutamate evoked changes in intracellular calcium have also resulted in considerable controversy in the literature. There are conflicting reports as to whether sigma receptor ligands exert their effects via actions on sigma receptors (Hayashi et al., 1995; Monnet et al., 2003) or non-specific interaction with other targets, in particular, NMDA receptors (Nishikawa et al., 2000; Kume et al., 2002). To some extent, analysis and interpretation of the results has been confounded by limitations in the pharmacological approaches used. For example, sigma receptors and NMDA receptors both bind PCP and related compounds (eg. MK-801) with high affinity (Sircar et al., 1987), and thus, such drugs cannot be used to discriminate between direct and indirect effects. Also, previous studies have not effectively used specific sigma receptor antagonists to confirm results.

Two distinct subtypes of sigma receptors have been identified on the basis of their pharmacological profile (Bowen et al., 1989; Quirion et al., 1992). Thus far, only the sigma-1 receptor has been cloned (Hanner et al., 1996), but the sigma-2 receptor has been shown to be a separate molecular entity (Langa et al., 2003). Sigma-1 receptor activation has been shown to prevent neuronal death associated with glutamate toxicity in cultured neurons (Kume et al., 2002; Takahashi et al., 1996) and to diminish infarct damage by decreasing neuronal nitric oxide production in vivo (Goyagi et al., 2001; Lesage et al., 1995). Sigma-1 also modulates the immune response by inhibiting TNF-α production in endotoxin-activated macrophages and acts as an anti-inflammatory agent by stimulating the expression of interleukin-10 during in vivo and in vitro ischemic simulations (Bourrie et al., 1996; Bourrie et al., 1995; Bourrie et al., 2002; Derocq et al., 1995). In contrast, less is known about sigma-2 receptors. However these receptors have been shown to regulate voltage-activated calcium and sodium channels in neurons (Zhang and Cuevas, 2002; Zhang and Cuevas, 2005), which is likely to enhance neuronal survival following stroke (Tanaka et al., 2002).

### SUMMARY OF INVENTION

Experiments were undertaken to determine if activation of sigma receptors in cultured cortical neurons modulates elevations in intracellular calcium observed in response to in vitro ischemia. Sigma receptors agonists that do not interact with NMDA receptors were shown to depress the peak amplitude of ischemia-induced calcium transients. Further experiments using sigma receptor-specific antagonists confirmed that the effects of sigma agonists are mediated by their actions on sigma receptors. Moreover, sigma receptor subtype-selective agonists showed that sigma-1 receptors are responsible for the observed depression of calcium elevations evoked by ischemia, whereas both sigma receptor subtypes regulate spontaneous calcium transients observed in cultured cortical neurons.

The ability of sigma-i and sigma-2 receptors to target different ion channels and different processes likely involved in neuronal demise following ischemia suggests that both should be targeted for stroke therapy. 1,3-di-o-tolyguanidine (DTG), a sigma ligand used here, has a high affinity for both sigma 1 and 2 receptors (Quirion et al., 1992). Activation of both sigma receptors will result in additive or synergistic neuroprotective and anti-inflammatory effects. DTG was administered subcutaneously to rats starting at 24 hours after permanent embolic middle cerebral artery occlusion (MCAO) and found that this treatment results in a significant decrease in infarction area.

According to the present invention there is provided a method of treating ischemic stroke comprising the step of administering a sigma receptor agonist to a patient in need thereof The sigma receptor agonist can include 1,3-di-o-tolyguanidine (DTG), carbetapentane, (+)- pentazocine, PRE-084, rimcazole, L-687,384, BD-737, JO-1784(igmesine). In certain aspects the sigma receptor agonist is a sigma-1 receptor agonist. In certain aspects the sigma receptor agonist is administered more than about three hours post-stroke. In further aspects the sigma receptor agonist is administered about twenty four hours post-stroke. In certain aspects the the sigma receptor agonist is a sigma-2 receptor agonist. The sigma-2 receptor agonist can include DTG, ifenprodil, ibogaine, CB 184, CB 64D, haloperidol and BIMU-8.

Also provided is a method of decreasing ischemia-induced elevations in intracellular calcium comprising the step of administering a sigma receptor agonist to a patient in need thereof. The sigma receptor agonist can include 1,3-di-o-tolyguanidine (DTG), carbetapentane, (+)- pentazocine, PRE-084, rimcazole, L-687,384, BD-737, JO-1784(igmesine). In certain aspects the sigma receptor agonist is a sigma-1 receptor agonist. In certain aspects the sigma receptor agonist is administered more than about three hours post-stroke. In further aspects the sigma receptor agonist is administered about twenty four hours post-stroke. In certain aspects the the sigma receptor agonist is a sigma-2 receptor agonist. The sigma-2 receptor agonist can include DTG, ifenprodil, ibogaine, CB 184, CB 64D, haloperidol and BIMU-8.

### BRIEF DESCRIPTION OF THE DRAWINGS

For a fuller understanding of the invention, reference should be made to the following detailed description, taken in connection with the accompanying drawings, in which:

FIG. 1 shows the increases [Ca^{2|}]ᵢ evoked by ischemia are dependent on the number of days the neurons have been in culture. (A) Change in [Ca²⁺]ᵢ ([Ca²⁺]ᵢ minus baseline [Ca²⁺]ᵢ) as a function of time for two neurons collected from the same neuronal isolation. Neurons were in culture for 3 (lighter black line) or 14 (heavier black line) days. Line above traces indicates application of chemical ischemia (0 glucose, 4 mM azide). (B) Bar graph of mean change in [Ca²⁺]ᵢ (Δ[Ca²⁺]ᵢ) obtained in response to chemical ischemia when neurons were held in culture for the indicated time points (n=8-34). Asterisks denote significant difference from 3 days (p<0.05) and dagger indicates significant difference from 21 days (p<0.05). (C) Change in [Ca²⁺]ᵢ as a function of time for a single neuron exposed to chemical ischemia for 2 mm (line above trace) in the absence (Control) and presence of 200 nM TTX (TTX). (D) Bar graph of mean change in [Ca²⁺]ᵢ obtained in response to chemical ischemia in the absence (Control) and presence of 200 nM TTX (TTX) (n=49). Asterisks denote significant difference from Control (p<0.001).

FIG. 2 shows that increases in [Ca²⁺]ᵢ evoked by ischemia are dependent on extracellular calcium and activation of IP₃-sensitive stores. (A) Bar graph of intracellular calcium levels recorded in the absence (0 Ca) and presence (Control) of external calcium. Asterisks denote significant difference from respective baselines (p<0.05), and pound symbol indicates significant difference from peak Control (p<0.05). (B) Bar graph of mean change in [Ca²⁺]ᵢ (Δ[Ca²⁺]ᵢ) obtained in response to chemical ischemia in the absence (Control; n=23) and presence of ryanodine (20 μM; n=9), or following preincubation in thapsigargin (10 μM, 45 mm, 23° C.; n=81). Asterisks indicate significant difference from respective baselines (p<0.05), and number symbol indicates significant difference from peak [Ca²⁺]ᵢ under control conditions and in the presence of ryanodine (p<0.05).

FIG. 3 shows that the sigma receptor-selective ligand, DTG, blocks elevations in [Ca²⁺]ᵢ evoked by in vitro ischemia. (A) [Ca²⁺]ᵢ as a function of time recorded from a single neuron during ischemic insults in the absence (Control) and presence of 50 μM DTG. (B) Mean peak [Ca²⁺]ᵢ measured during an ischemic episode in the absence and presence of 50 μM DTG (n=13). Asterisks indicate significant difference from Control (p<0.001).

FIG. 4 shows that the inhibition of sigma receptors by metaphit blocks DTG-mediated suppression of ischemia-evoked increases in [Ca²⁺]ᵢ. (A) [Ca²⁺]ᵢ as a function of time recorded from 4 different neurons during ischemic insults in the absence (Control) and presence of 50 μM DTG (DTG) and following preincubation in 50 μM metaphit (MET) or in the presence of DTG following metaphit preincubation (MET+DTG). (B) Mean peak [Ca²⁺]ᵢ measured during ischemic episodes under the indicated conditions (n=19-29). Asterisks, number symbol, and dagger denote significant difference from Control, DTG alone (DTG), and metaphit alone (MET) groups, respectively (p<0.05). (C) Change in [Ca²⁺]ᵢ recorded in the presence of DTG in control cells (DTG; n=13) and cells preincubated in metaphit (MET+DTG; n=27) normalized to the mean Δ[Ca²⁺]ᵢ recorded for the control of each test group (eg. Control and MET). Asterisks indicate significant difference (p<0.001).

FIG. 5 shows that the concentration-dependent relief of DTG inhibition of ischemia-induced transient elevations in cytoplasmic Ca²⁺ by the sigma receptor antagonist, BD-1047. (A) typical traces of [Ca²⁺]ᵢ recorded from three E18 neurons in response to ischemia under control conditions (heavier solid black line), with 100 μM DTG (lighter solid black line) or with 100 μM DTG and 10 μM BD-1047 (dotted black line). (B) Bar graph of relative changes in [Ca²⁺]ᵢ for neurons exposed to ischemia under control conditions (Control, n=77 cells), with 100 μM DTG (n=83 cells), with 100 μM DTG and 1 μM BD-1047 (DTG+1 μM BD, n=93 cells) and with 100 μM DTG plus 10 μM BD-1047 (DTG+10 μM BD, n=145 cells). The asterisks denote significant difference (p<0.05) from control and number symbols denote significant difference (p<0.05) from 100 μM DTG treatment.

FIG. 6 shows that the activation of sigma 1 receptors in cortical neurons depresses ischemia-induced elevations in [Ca²⁺]ᵢ. (A) [Ca²⁺]ᵢ as a function of time recorded from three different neurons during an ischemic insult in the absence (Control) and presence of carbetapentane (CBP) at the indicated concentrations. (B) Mean peak [Ca²⁺]ᵢ measured during ischemic episodes with the indicated concentrations of carbetapentane (n=57-107). Solid line is a best fit to the data using a single-site Langmuir-Hill equation with an IC50 value of 13.3 μM and a Hill coefficient of 0.8.

FIG. 7 shows that the sigma-2 receptor agonist, ibogaine, fails to block ischemia-induced elevations in [Ca²⁺]ᵢ in cortical neurons. (A) Representative traces of [Ca²⁺]ᵢ recorded from two neurons in the absence (Control) and presence of 100 μM ibogaine (IBO). (B) Mean peak [Ca²⁺]ᵢ measured in cortical neurons during ischemic episodes in the absence (Control) and presence of ibogaine at the indicated concentrations (n=4-9).

FIG. 8 shows that concentration-dependent reduction of ischemia induced transient elevations in cytoplasmic Ca²⁺ by the sigma-1 receptor agonists (+)-pentazocine and PRE-084. (A) typical traces of [Ca²⁺]ᵢ recorded from E18 neurons in response to either ischemia (heavier solid black line), or ischemia in the presence of (+)-pentazocine (PTZ; 10 μM, lighter solid black line; 100 μM, dashed black line). (B) bar graph of relative changes in [Ca²⁺]ᵢ for neurons exposed to ischemia (Control, n=104), ischemia and 10 μM (+)-pentazocine (10 μM PTZ, n=90) and 100 μM (+)-pentazocine (100 μM PTZ, n=104). The asterisks denote significant difference (p<0.05) from control. (C) Representative traces of [Ca²⁺]ᵢ recorded from three E18 neurons in response to either ischemia under control conditions (heavier solid black line), with 10 μM PRE-084 (lighter solid black line) or with 100 μM PRE-084 (dotted black line). (D) bar graph of relative changes in [Ca²⁺]ᵢ for neurons exposed to ischemia in the absence (Control, n=432 cells), and presence of 10 μM PRE-084 (10 μM PRE, n=110 cells) and 100 μM PRE-084 (100 μM PRE, n=122 cells). The asterisks denote significant difference (p<0.05) from control and the number symbols denote significant difference (p<0.05) from azide and 10 μM PRE-084 treatment.

FIG. 9 shows that spontaneous Ca²⁺ transients are blocked by sigma receptor agonists. (A) typical traces of [Ca²⁺]ᵢ recorded from a single neuron prior to (Control), during and following washout (Wash) of bath applied 100 μM DTG (i.), 50 μM ibogaine (ii., IBO), and 100 μM carbetapentane (iii., CBP). Bar graph of mean frequency of events detected under conditions in (A) using the sigma ligands DTG (B, n=59), ibogaine (C, n=77), and carbetapentane (D, n=21). Asterisks denote significant difference (p<0.05) from respective controls.

FIG. 10 shows the effects of sigma receptor ligands on MCAO survival rates. Survival rate of sham control injected with 15 mg/kg of DTG (Sham+DTG 15), and MCAO rats under the indicated conditions. Rats subjected to MCAO received vehicle alone (No Drug), DTG at 15 mg/kg or 30 mg/kg (DTG 15 QD, DTG 30 QD, DTG 30 BID), 10 mg/kg BD 1047 (BD 1047 10 QD), or 10 mg/kg BD 1047 and 30 mg/kg DTG (BD 1047+DTG 30 QD). QD and BID denote once daily or twice daily administration of the compounds, respectively. Asterisks indicate significant difference from DTG 30 BID. All injections commenced at 24 hr and continued to 72 hr post surgery.

FIG. 11 shows that the DTG treatment reduced Fluoro-Jade staining when administered 24 hours post-MCAO. Photomicrographs of brain sections were taken from MCAO rats in the absence (MCAO; panels A and B) and presence of DTG treatment (MCAO+DTG 15 mg/kg; panels C and D). Coronal sections were collected at the level of the cortical/striatal (Striatum; panels A and C) or cortical/hippocampal (Hippocampus; panels B and D) regions. Fluoro-Jade staining appears as bright green coloration (lighter area) and is indicative of area damaged by the ischemic insult. Scale bars represent 5 mm at a magnification of 12.5×.

FIG. 12 shows the quantification of DTG-elicited reduction in post-MCAO Fluoro-Jade staining. Fluoro-Jade staining was analyzed in the cortical/striatal (Striatum, black bars) and cortical/hippocampal (Hippocampus, gray bars) regions of sham controls (Sham), rats subjected to MCAO receiving only vehicle (MCAO) and rats subjected to MCAO receiving 15 mg/kg DTG 24 hours post-surgery (DTG). Bars represent mean±standard error. Asterisks denote significant difference from respective areas of sham control and DTG treated rats, and was determined using a two way ANOVA followed by post hoc analysis with a Dunn's Test for multiple group comparison (p<0.01).

FIG. 13 shows that DTG treatment increases NeuN immunostaining when administered 24 hours post-MCAO. Photomicrographs of brain sections were taken from MCAO rats in the absence (MCAO; panels A-C) and presence of DTG treatment (MCAO+DTG 15 mg/kg; panels D-F). Coronal sections were collected at the level of the ipsilateral cortical/striatal (Striatum; panels A, C, D and F) or cortical/hippocampal (Hippocampus; panels B and D) regions. Boxes in panels A and D indicate field of view shown at higher magnification in panels C and F, respectively. Individually stained nuclei from neurons of the cortical/striatal regions were visualized using higher magnification (200×; panels C and F). NeuN staining appears as dark brown coloration and is indicative of viable neurons. Scale bars (A, B, D, and E) represent 30 μm at a magnification of 40×. Scale bars for C and F represent 50 μm at 200×.

FIG. 14 shows that DTG treatment at delayed time points significantly increases the number of NeuN positive cells following MCAO. The number of NeuN positive neurons detected in ipsilateral (IPSI) and contralateral (CONTRA) hemispheres of cortical/hippocampal (Hippocampus) and cortical/striatal (Striatum) regions of sham control rats (SHAM, white bars), MCAO rats injected with vehicle alone (MCAO, black bars) and MCAO rats treated with 15 mg/kg of DTG (DTG, gray bars). Bars represent mean count±standard error. Statistical significance from equivalent region in the contralateral hemisphere is indicated by the asterisks, and from the ipsilateral hemisphere of SHAM and DTG groups by the pound symbol (p<0.01 for both). Statistical significance was determined using a two way ANOVA followed by post hoc analysis with a Tukey Test for multiple group comparison.

FIG. 15 shows that DTG decreases the intensity of GFAP immunostaining surrounding the infarct zone when administered 24 hours post-MCAO. Photomicrographs of brain sections were taken from MCAO rats in the absence (MCAO; panels A-C) and presence of DTG treatment (MCAO+DTG 15 mg/kg; panels D-F). Coronal sections were collected at the level of the ipsilateral cortical/striatal (Striatum; panels A, C, D and F) or cortical/hippocampal (Hippocampus; panels B and D) regions. Boxes in panels A and D indicate field of view shown at higher magnification in panels C and F, respectively. Reactive astrocytes showing high levels of GFAP were observed in the cortical/striatal region using higher magnification (200×; panels C and F). GFAP staining appears as dark brown coloration. Scale bars in panels A, B, D and E represent 30 μm at a magnification of 40×, and in panels C and F represent 50 μm at 200×.

FIG. 16 shows that DTG treatment decreases Isolectin IB4 binding when administered 24 hours post-MCAO. Photomicrographs of coronal brain sections were taken from the ipsilateral cortical/striatal region of MCAO rats in the absence (MCAO; panels A and B) and presence of DTG (15 mg/kg) treatment (MCAO+DTG; panels C and D) as well as sham controls treated with DTG (SHAM; panels E and F). Boxes in panels A, C and E indicate field of view shown at higher magnification in panels B, D and F, respectively. Individual labeled activated microglia and/or macrophages from cortical/striatal regions were visualized using higher magnification (200×; panels B, D and F). Isolectin IB4 labeling appears as bright green coloration (lighter area). Scale bars (A, C, E) represent 20 μm at 40×, and scale bars (B, D, F) represent 50 μm at 200×.

FIG. 17 shows a representation of the pathobiology of ischemic stroke. Disregulation of intracellular calcium is a central component in neuronal death resulting from ischemic injury in the CNS.

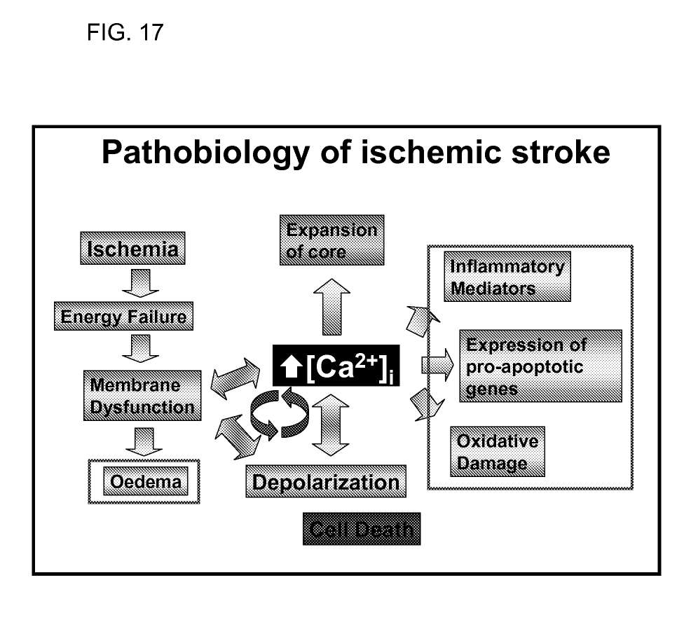

FIG. 18 shows a representation of sigma-1 and sigma-2 receptors. Two sigma receptor subtypes have been identified on the basis of pharmacology. Sigma-1 receptors have a higher affinity for (+)-stereoisomer of benzomorphans, while sigma-2 receptors have a higher affinity for ibogaine. 1,3-Di-o-tolylguanidine (DTG) is a nonselective sigma agonist.

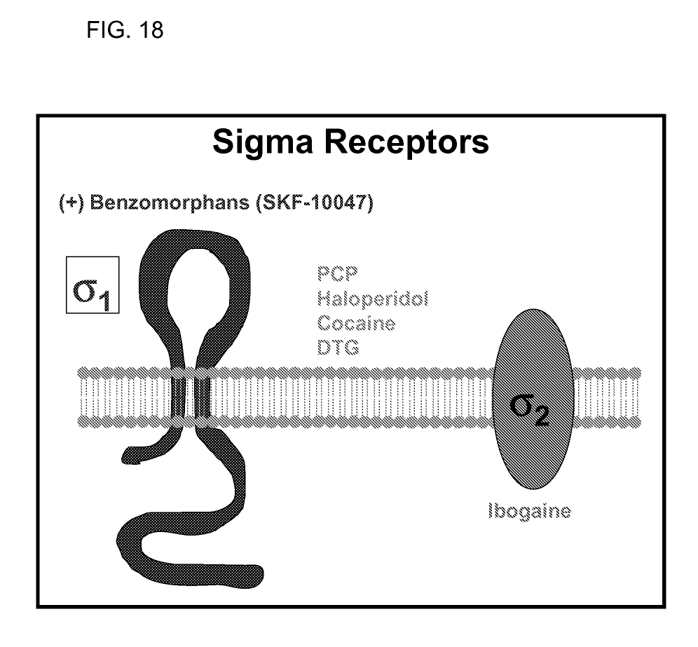

FIG. 19 shows a representation of the pleiotropic effects of sigma receptors. Stimulation of sigma receptors depresses activity in cortical neurons by affecting multiple presynaptic and postsynaptic targets. The relationship between sigma receptors and ASICs remains unknown.

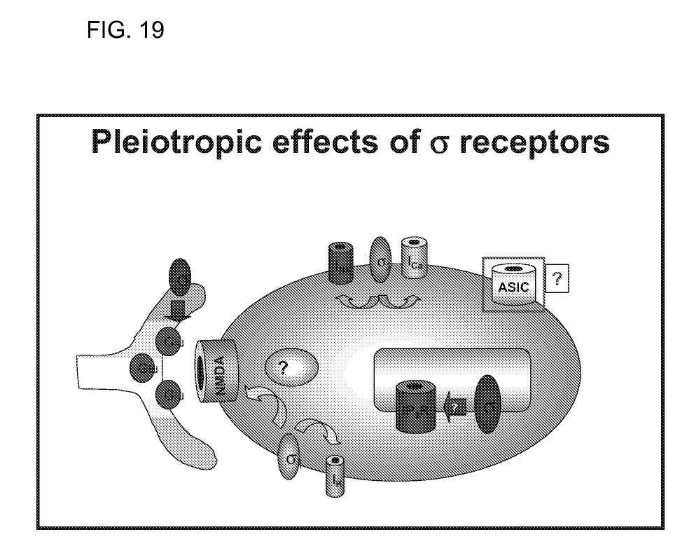

FIG. 20 shows that sigma receptor activation blocks ischemia-induced elevations in [Ca^{2|}]ᵢ. (A) [Ca^{2|}]ᵢ as a function of time recorded from 4 different neurons during ischemic insults in the absence (Control) and presence of 50 μM DTG (DTG) and following preincubation in 50 μM metaphit (MET) or in the presence of DTG following metaphit preincubation (MET+DTG). (B) Mean peak [Ca²⁺]ᵢ measured during ischemic episodes under the indicated conditions (n=19-29). Asterisks, number symbol, and dagger denote significant difference from Control, DTG alone (DTG), and metaphit alone (MET) groups, respectively (p<0.05). (C) Change in [Ca²⁺]ᵢ recorded in the presence of DTG in control cells (DTG; n=13) and cells preincubated in metaphit (MET+DTG; n=27) normalized to the mean Δ[Ca²⁺]ᵢ recorded for the control of each test group (eg. Control and MET). Asterisks indicate significant difference (p<0.001). Sigma receptor activation depresses ischemia-induced elevations in [Ca²⁺]ᵢ. Inhibition of sigma receptors enhances ischemia-evoked increases in [Ca²⁺]ᵢ.

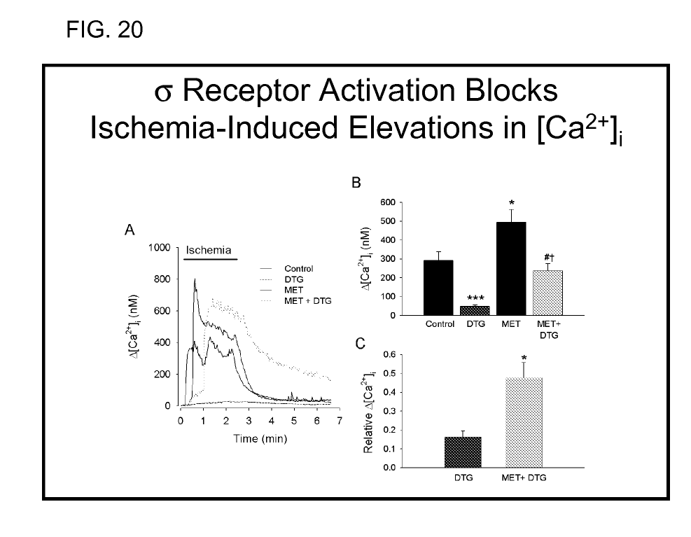

FIG. 21 shows that DTG inhibits low-pH evoked transient increases in [Ca²⁺]ᵢ.(A) Representative traces of [Ca²⁺]ᵢ as a function of time recorded from a single neuron during acidosis in the absence (Control) and presence of 100 μM DTG (DTG). (B) Mean peak [Ca²⁺]ᵢ measured during acidic insult under the indicated conditions (n=31). Asterisk denotes significant difference from Control and DTG groups, (p<0.05). The sigma receptor agonist, DTG, inhibits the function of ASIC channels in cortical neurons.

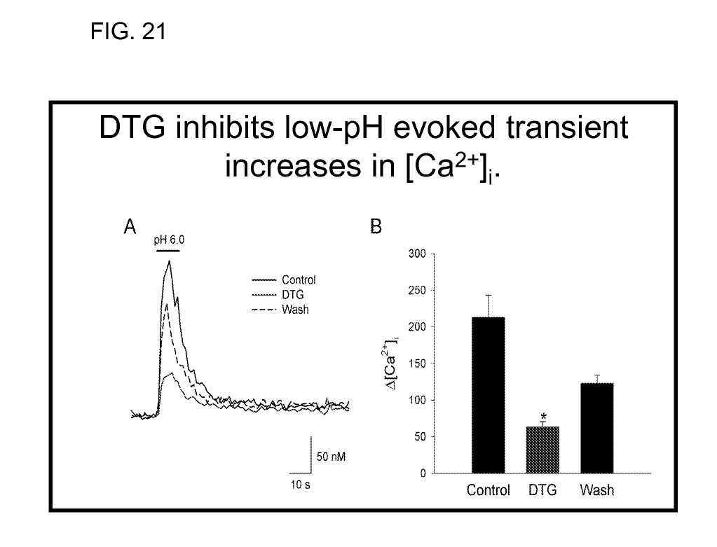

FIG. 22 shows that pentazocine attenuates ASIC-mediated transient increases in [Ca²⁺]ᵢ. (A) Representative traces of [Ca²⁺]ᵢ as a function of time recorded from a single cell during acidosis in the absence (Control) and presence of 50 μM Pentazocine (PTZ). (B) Mean peak [Ca²⁺]ᵢ measured during acidic insult under the indicated conditions (n=4). Asterisk denotes significant difference from Control and PTZ groups, (p<0.05). Pentazocine, at concentrations known to affect sigma receptors, reversibly decreases the low-pH evoked increases in [Ca²⁺]ᵢ by 50%. Activation of sigma receptors modulates the function of ASIC channels.

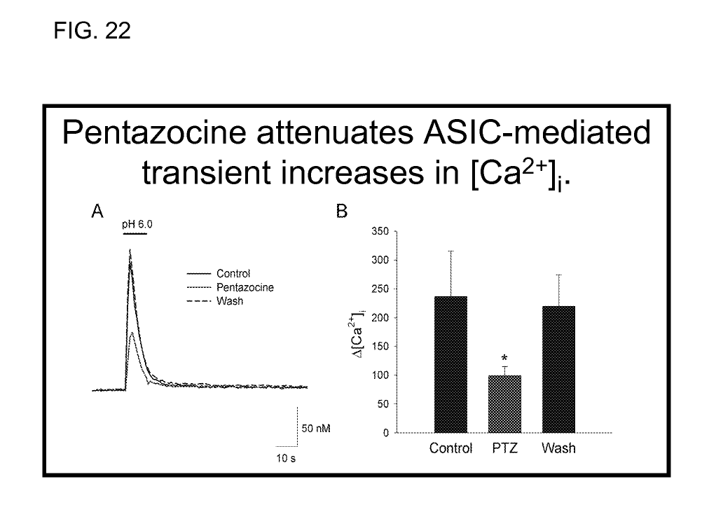

FIG. 23 shows that ibogaine depresses ASIC-mediated transient increases in [Ca²⁺]ᵢ. (A) Representative traces of [Ca²⁺]ᵢ as a function of time recorded from a single cell during acidosis in the absence (Control) and presence of 50 μM ibogaine. (B) Mean peak [Ca²⁺]ᵢ measured during acidosis under the indicated conditions (n=18). Asterisk denotes significant difference from Control and Ibogaine groups, (p<0.05). Ibogaine, a sigma-2 receptor agonist, reversibly decreases the low-pH evoked increases in [Ca²⁺]ᵢ by 70%. Sigma-2 receptors modulate ASICs in cortical neurons. Rapid focal application of low pH saline solution (pH 6.0) evoked transient elevations in [Ca²⁺]ᵢ in >80% of cortical neurons tested (n=220). The sigma receptors agonists, DTG, Ibogaine and Pentazocine reversibly reduce the low pH evoked increases in [Ca²⁺]ᵢ. The rank order potency ibogaine >(+) pentazocine is consistent with sigma-2 receptor modulation of ASICs. Our findings raise the possibility that sigma receptor-mediated neuroprotection is in part due to the inhibition of ASICs.

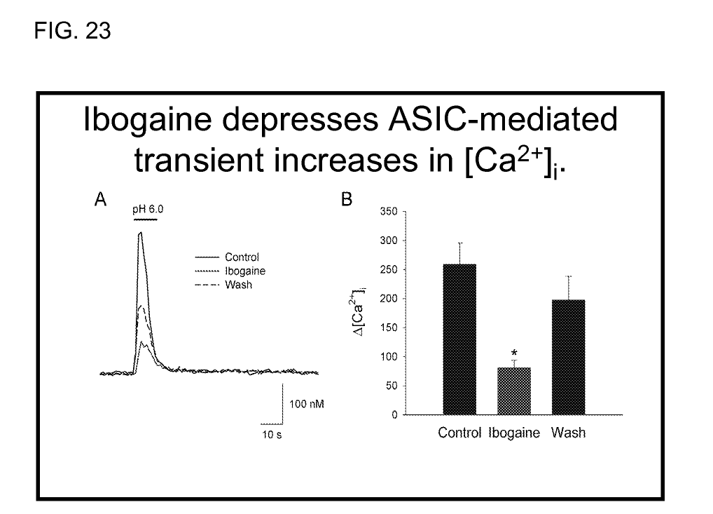

### DETAILED DESCRIPTION OF THE PREFERRED EMBODIMENT

We report that activation of sigma-1 receptors depress ischemia-induced dysregulation of intracellular calcium in cultured cortical neurons. Pharmacological studies using sigma selective agonists and antagonists clearly show that the effects of the sigma ligands are mediated by actions on sigma receptors and are not the result of modulation of NMDA receptors by the drugs. Our data also indicate that tonic activation of sigma receptors or activation of sigma receptors upon induction of ischemia via an endogenous mechanism diminishes ischemia-induced elevations of intracellular calcium. Finally, our findings also demonstrate that while sigma-2 receptors do not appreciably influence ischemia-induced changes [Ca²⁺]ᵢ, these receptors can decrease spontaneous calcium transients observed in cultured cortical neurons.

Previous studies have shown that the sigma ligands (+)SKFIOO47 (10 μM) and haloperidol (10 μM), but not carbetapentane (100 μM) or DTG (100 μM), inhibit calcium elevations evoked by glutamate application (Kume et al., 2002; Klette et al., 1995). Our studies show that DTG effectively blocks ischemia-induced elevations in [Ca²⁺]ᵢ at concentrations that have little or no effect on changes in [Ca²⁺]ᵢ elicited by direct glutamate application (Klette et al., 1995). In contrast, similar concentrations DTG (65 μM) have been shown to stimulate sigma receptor modulation of electrical activity in frog pituitary melanotrope cells (Soriani et al., 1998). Low concentrations of carbetapentane (10 μM) were shown to inhibit-P50% of the peak ischemia-induced increases in [Ca²⁺]ᵢ, whereas 10-fold higher concentrations of this sigma receptor agonist failed to block glutamate-induced increases in [Ca²⁺]ᵢ (Kume et al., 2002). Taken together, these data suggest that the effects of sigma ligands on ischemia-induced increases in [Ca²⁺]ᵢ cannot be explained by the actions of these drugs on metabotropic and ionotropic glutamate receptors (eg. NMDA receptors) alone, and are the result of these drugs acting on sigma receptors.

The role of sigma receptors in the depression of ischemia-elicited increases in [Ca²⁺]ᵢ is further supported by experiments using the sigma receptor selective antagonists metaphit and BD-1047. Our laboratory has previously shown that metaphit is an irreversible inhibitor of sigma receptors, and that preincubation of neurons in metaphit inhibits sigma-1 and sigma-2 receptor block of K⁺ and Ca²⁺ channels, respectively (Zhang and Cuevas, 2002; Zhang and Cuevas, 2005). It is important to note that while metaphit, a PCP analog, can attenuate phencyclidine-induced antagonism of NMDA responses, it does not have any direct effects on NMDA mediated responses even at concentrations significantly greater than those used here (Wang and Lee, 1991). BD-1047 was also shown to block DTG-mediated inhibition of ischemia related dysregulation of [Ca²⁺]ᵢ at concentrations selective for sigma receptors (Matsumoto et al., 1995). BD-1047 has also been used previously to show that sigma receptors mediate DTG-evoked hypothermia and the effects of cocaine on conditioned place preference (Rawis et al., 2002; Romieu et al., 2004).

The observation that the sigma-1 selective agonists (+)-pentazocine, PRE-084, and carbetapentane, but not the sigma-2-selective agonist, ibogaine, mimicked the effects of DTG on ischemia-induced elevations in [Ca^{2|}]ᵢ, indicates that sigma-1 receptors are responsible for the observed effects. Studies have shown that the affinity of sigma-1 receptors for carbetapentane is >30-fold greater than that of sigma-2 receptors, whereas the affinity of sigma-2 receptors for ibogaine is >40-fold greater than that of sigma-1 receptors (Hirata et al., 2006; Vilner and Bowen, 2000). The calculated IC50 for carbetapentane inhibition of ischemia-evoked increases in [Ca²⁺]ᵢ (13 μM) is similar to values reported for carbetapentane inhibition of epileptiform activity via sigma receptors in rat hippocampal slices (38 μM) (Thurgur and Church, 1998). Furthermore, the affinity of sigma-1 receptors for (+)-pentazocine is ˜2000-fold greater than for ibogaine, whereas the affinity of sigma-2 receptors for ibogaine is ˜6-fold higher than for (+)-pentazocine (Vilner and Bowen, 2000). Here it is shown that 10 μM (+) pentazocine blocked ˜60% of ischemia-evoked increases in [Ca²⁺]ᵢ, which is consistent with the IC50 for (+) pentazocine inhibition of delayed outwardly rectifying K^{|} channels (37 μM) and voltage-gated K⁻ channels (42 μM) via sigma-1 receptors in frog pituitary melanotrophs and rat intracardiac neurons, respectively (Soriani et al., 1998; Zharig and Cuevas, 2005). In contrast, in rat intracardiac neurons sigma-2 receptors inhibit voltage-gated Ca²⁺, and the IC50 reported for ibogaine is 31 μM (Zhang and Cuevas, 2002). Even at 3-fold higher concentrations, ibogaine failed to affect the ischemia-induced elevations in [Ca²⁺]ᵢ. Thus, the attenuation of elevations in [Ca²⁺]ᵢ is primarily the result of sigma ligands acting on sigma-1 receptors in cortical neurons.

The mechanism by which sigma-1 receptors modulate ischemia-induced elevations in [Ca²⁺]ᵢ remains to be determined. However, several factors are likely involved due to the complex nature of the responses observed in our system. The dependance of the peak amplitude of ischemia-induced elevations in [Ca²⁺]ᵢ on the number of days the neurons are in culture coincides with the development of synapses in this preparation, suggesting that the phenomenon involves synaptic transmission. Also consistent with this hypothesis are the observations that either application of TTX or removal of extracellular calcium significantly depressed [Ca²⁺]ᵢ responses. One possibility is that sigma-1 receptor activation is decreasing glutamate release under these conditions. Studies have shown that DTG can decrease glutamate release evoked by oxygen and glucose deprivation from hippocampal brain slices (Lobner and Lipton, 1990). Alternatively, sigma-1 receptors may be modulating postsynaptic receptors, and inhibiting neurotransmission. It has been suggested that sigma receptors may block calcium responses associated with activation of both ionotropic and metabotropic glutamate receptors (Klette et al., 1997). The fact that sigma-1 receptor activation can eliminate the elevations in [Ca²⁻]ᵢ suggests that they are blocking calcium entry through the plasma membrane and calcium release from intracellular stores, consistent with inhibition of both glutamate receptor types.

The fact that removal of extracellular calcium failed to abolish the [Ca²⁺]ᵢ responses suggest that these are not exclusively dependent on neurotransmission, and that a component of the elevations in [Ca²⁺]ᵢ may be due to calcium leak from intracellular stores as a result of metabolic inhibition. This observation is consistent with previous studies on hippocampal slices which showed that oxygen-glucose deprivation can cause increases in [Ca^{2|}]ᵢ even in Ca^{2|}-free and Ca^{2|}-containing solutions (Ebine et al., 1994). Thus, activation of sigma receptors must also block calcium release from these stores, since sigma receptor activation inhibited ischemia elicited elevations in [Ca²⁺]ᵢ to a greater extent than either removal of extracellular calcium or TTX alone. Various studies have shown that intracellular organelles that release calcium under ischemic conditions, such as the endoplasmic reticulum and mitochondria (Mattson, 2000), are rich in sigma receptors (Hayashi and Su, 2003; McCann et al., 1994). While the function of sigma receptors on these intracellular sites is not fully understood, they do appear to be capable of regulating calcium release from at least the endoplasmic reticulum (Hayashi et al., 2000). An interesting observation reported here is that inhibition of sigma receptors resulted in elevations in basal [Ca²⁺] and potentiated the increases in [Ca²⁺] evoked by ischemia. Thus, sigma receptors appear to be involved in calcium homeostasis in cortical neurons under control conditions. Previous studies have shown that sigma receptors can modulate various plasma membrane calcium channels, including voltage-gated Ca²⁺ channels and NMDA receptors (Hayashi et al., 1995; Zhang and Cuevas, 2002), and can regulate phosphatidylinositide turnover (Hayashi et al., 2000). These findings have led to the theory that one of the critical cellular functions of sigma receptors is the regulation of intracellular calcium levels (Hayashi et al., 2000; Monnet, 2005). The observations reported here lend further support to this theory. Moreover, it appears that changes in intracellular calcium are modulated by both sigma-1 and sigma-2 receptors. While sigma-1 receptors affect the ischemia-induced changes in [Ca²⁺], both sigma receptor subtypes can depress spontaneous calcium transients observed in cultured cortical neurons. Low concentrations of DTG and ibogaine depressed the genesis of these calcium transients, consistent with a sigma-2 mediated effect. However, the fact that carbetapentane also inhibited these spontaneous Ca transients suggests that sigma-1 receptors may also regulate this phenomenon. It remains to be determined if sigma-1 and sigma-2 receptors affect this spontaneous activity via actions on identical targets (e.g. ion channel, calcium store, etc.). These spontaneous increases in [Ca^{2|}] have a frequency that is similar to bursts of spontaneous action potentials observed in our preparation (data not shown), but the exact source and triggering mechanism for these calcium transients remains to be determined. Previous studies have reported these synchronous calcium transients, and they appear to be correlated with bursts of electrical activity, axon outgrowth and synaptic development (Robinson et at., 1993; Tang et al., 2003).

In summary, the studies show that sigma-1 receptors mediate the depression of ischemia-induced elevations in [Ca²⁺]. Findings reported here clearly establish that sigma ligands can affect cellular function during ischemia and the concomitant excitotoxicity by acting on sigma receptors, rather than through non-specific effects on other molecular targets. Given that intracellular calcium dysregulation greatly contributes to the demise of cortical neurons following ischemic injury, sigma receptor-mediated neuroprotection is likely due in part to the preservation of intracellular calcium homeostasis. Thus, sigma receptors are a viable target for neuroprotection following ischemia and possibly other neurodegenerative diseases involving excitotoxicity.

The application of DTG 24 hr after stroke injury significantly decreases neurodegeneration in rats subjected to MCAO. Moreover, it was observed that the administration of this sigma ligand depresses the inflammatory response evoked by the ischemic insult. The role of sigma receptors in the effects of DTG were supported by the observation that the sigma-selective antagonist, BD-1047, worsened stroke outcomes. Thus, our data support the hypothesis that the window for treatment of stroke extends beyond the limitations of the currently available therapy, and that sigma receptors are a viable target for stroke therapy at delayed time points.

Two lines of evidence presented here confirm that DTG is neuroprotective when administered 24 hr post stroke. First, DTG significantly decreased Fluoro-Jade staining, which is consistent with reduced neurodegeneration, by >85% relative to untreated MCAO animals. Second, the number of surviving cells detected in the infarct zone with the neuron specific marker, NeuN, was increased by >83% in DTG treated animals relative to MCAO-only animals. Moreover, the number of viable cells observed in the ischemic region of DTG treated animals was similar to the number of cells present in equivalent regions of sham controls. Previous studies on the neuroprotective properties of sigma receptor activation following MCAO have focused on a transient ischemia model (1-2 hr occlusion) and have commenced intravenous application of sigma-1 selective ligands, such as 4-phenyl-1-(4-phenylbutyl)piperidine (PPBP), 1-2 hr following reperfusion ((Takahashi et al., 1996). Brain sections taken from animals treated with PPBP showed 40% decrease in neurodegeneration (Takahashi et al., 1996). Unlike observations reported here, some studies suggest that acute application of PPBP following MCAO fails to decrease infarct injury in the striatum of rats (Harukuni et al., 2000). The sigma-1 ligand, JO 1784, has also been shown to be effective for decreasing brain injury following global ischemia when applied >1 hr following the ischemic insult (O'Neill et al., 1995). Our studies, however, indicate that DTG can exert its neuroprotective properties even when treatment begins 24 hr post-stroke.

The dose of DTG used here to diminish stroke injury without compromising survival rates (15 mg/kg) is comparable to doses previously used of this sigma ligand to elicit sigma receptor-mediated effects in vivo, such as hypothermia (1-30 mg/kg) and antinociception (10-20 mg/kg)(Kest et al., 1995; Rawls et al., 2002). The mechanism(s) by which higher concentrations of DTG (30 mg/kg) increase mortality following MCAO remains to be determined. However, sigma receptors have been shown to modulate cardiac function directly by acting on cardiac muscle and to regulate neurons that mediate autonomic control of the heart (Novakova et al., 1998; Zhang and Cuevas, 2002; Zhang and Cuevas, 2005). Given the fact that cardiovascular abnormalities, including cardiac arrhythmias, are associated with stroke (Klingelhofer and Sander, 1997), high concentrations of DTG may exert their deleterious effects via enhancing cardiac dysfunction following stroke injury.

The inflammatory response that occurs in the central nervous system following injury, such as that produced by ischemic stroke, plays a significant role in enhancing neurodegeneration. The sequence of events leading to cerebral inflammation after acute focal ischemia begins with the rapid activation of microglia within 24 hours of the insult (Schroeter et al., 1997). This event is followed by systemic macrophage infiltration through the compromised blood brain barrier at 48 hours post-stroke (Schroeter et al., 1997). Subsequently, reactive astrocytes begin to release glutamate, nitric oxide, TNF-α, and other factors, which results in increased inflammation contributing to delayed neuronal death (Swanson et al., 2004). Based on data presented here, DTG blunts the glia-mediated inflammatory response evoked by MCAO. Three mechanisms may account for the observed sigma receptor-mediated anti-inflammatory response. First, direct neuroprotection by DTG administration may result in reduced production and release of signaling molecules which trigger the pro-inflammatory response. Various in vitro studies have shown that stimulation of sigma receptors decreases neuronal death in response to hypoxia and glutamate excitotoxicity (DeCoster et al., 1995; Lockhart et al., 1995). Second, stimulation of sigma receptors on cells involved in inflammation in the CNS may dampen this response. Sigma receptor agonists like SSR125329A and SR 31747 decrease inflammation by inducing the release of anti-inflammatory cytokines such as interleukin-10 and by decreasing the release of pro-inflammatory cytokines such as TNF-α (Bourrie et al., 2002) (Derocq et al., 1995). Results from our laboratory have show that DTG treatment inhibits the production of nitric oxide and TNF-α from cultured microglia in response to LPS (Hall et al., 2005), suggesting that DTG could directly inhibit the inflammation associated with ischemic injury in our in vivo model of stroke. Finally, the anti-inflammatory effects of DTG may involve a combination of both decreased signaling due to neuroprotection and an arrest of the endogenous inflammatory response. Regardless of the mechanisms involved, the anti-inflammatory effects of sigma receptor stimulation is in part responsible for the DTG-evoked depression of delayed neuronal death induced by ischemia.

Few treatments have shown success in expanding the therapeutic window for stroke. One approach that has exhibited promise in treating stroke at delayed time points is intravenous infusion of HUCBC. HUCBC have been shown to effectively decrease infarct volume by 50-80% when injected 24-48 hr in rats following MCAO (Newcomb et al., 2005; Vendrame M et al., 2005; Vendrame et al., 2004). Like DTG, HUCBC treatment shows both neuroprotective and anti-inflammatory properties. This observation is consistent with our hypothesis that both of these properties are essential for effective treatment of stroke at delayed time points. Moreover, HUCBC are potentially activating sigma receptors upon transfusion through release of neurosteroids. Umbilical plasma has been shown to contain more than double the concentration of neuroactive steroids when compared to adult plasma (Hill et al., 2000). Neurosteroids have been shown to have high affinity for sigma receptors and have been proposed as the endogenous ligand for these receptors (Maurice, 2004).

The specific sigma receptor subtype mediating the neuroprotective effects of DTG remains to be identified. However, data collected in our laboratory and by other investigators suggests that both sigma-1 and sigma-2 receptors are likely to be involved in the enhanced neurosurvival reported here. Sigma-1 receptors have been shown to block both voltage-gated K^{|} channels and NMDA receptors (Aydar et al., 2002; Zhang and Cuevas, 2005)(Nuwayhid and Werling, 2003). Both of these ion channel types have been linked to the neuronal damage which is produced by ischemic stroke (Bonde et al., 2005; Gido et al., 1997). Our laboratory has now shown that sigma-1 receptors can decrease calcium elevations elicited by ischemia in neurons in vitro (Zhang and Cuevas, 2005), which would also provide neuroprotection (Mattson et al., 2000). Sigma-2 receptors have been shown to inhibit voltage-gated Ca²⁺ channels (Zhang and Cuevas, 2002), and the inhibition of these channels is neuroprotective (Kristian and Siesjo, 1997). In addition to regulating ion channels, sigma receptors are likely to modulate other processes that contribute to neuronal injury following stroke. Sigma-1 and sigma-2 receptors have been detected in lipid rafts in various cell types, and are likely to modulate cytokine signaling involving these microdomains (Hayashi and Su, 2005). Both sigma receptor subtypes have also been implicated in the regulation of apoptosis in tumor cell lines, with sigma-1 receptors inhibiting apoptosis and sigma-2 receptor promoting apoptosis in these cells (Crawford and Bowen, 2002; Spruce et al., 2004). However, the role of sigma receptors in regulation of cell survival in native non-tumor cells remains to be established.

In conclusion, the results clearly demonstrate that the sigma receptor-selective agonist, DTG, can enhance neuronal survival when administered 24 hr after an ischemic stroke. Conversely, application of the sigma receptor antagonist, BD-1047, decreases survival rates following MCAO. Thus, the studies identify sigma receptors as one of the first potential targets for expanding the therapeutic window beyond that provided by the currently available pharmacological treatments. In addition, the efficacy of sigma receptors for stroke treatment at delayed time points is likely the result of combined neuroprotective and anti-inflammatory properties of these receptors.

Materials and Methods

Preparation

The effects of sigma receptors on ischemia-induced changes in intracellular calcium concentrations were studied in cultured cortical neurons from embryonic (E18) rats. Dams were euthanatized by decapitation, uterus removed, and embryos dissected out and placed in isotonic buffer containing (in mM): 137 NaCl, 5 KCl, 0.2 NaH₂PO₄, 0.2 KH₂PO₄, 5.5 glucose, 6 sucrose (pH 7.4 with NaOH). Cortex were excised and minced, and tissue digested in isotonic buffer containing 0.25% trypsin/EDTA for 10 min at 37° C. and added to 3× volume of high glucose culture media (Dulbecco's Modified Eagle Media; Invitrogen, Inc., Carlsbad, Calif.), 10% (v/v) fetal calf serum, 100 U/ml penicillin and 0.1 mg/ml streptomycin. Cells were counted on a hemocytometer, plated (0.5×10⁶ cells) on 18 mm coverslips coated with poly-l-lysine, and incubated at 37° C. under a 95% air, 5% CO₂ atmosphere. After 24 hr the media was replaced with Neurobasal (Invitrogen) medium supplemented with B27 (Invitrogen) and 0.5 mM 1-glutamine to limit astrocyte proliferation in the cultures. Cells were used after 14-21 days in culture.

Microfluorometric Measurements

Intracellular free-calcium was measured using the calcium sensitive dye, fura-2 as previously described (DeHaven and Cuevas, 2004). Cells plated on coverslips were incubated for 1 hour at room temperature in physiological saline solution (PSS) consisting of (in mM): 140 NaCl, 3 KCl, 2.5 CaCl₂, 1.2 MgCl₂, 7.7 glucose and 10 HEPES (pH to 7.2 with NaOH), which also contained 1 μM of the membrane permeable ester form of fura-2, acetoxymethylester (fura-2 AM) and 0.1% dimethyl sulfoxide (DMSO). The coverslips were then washed in PSS (fura-2 AM free) prior to the experiments being carried out. All solutions were applied via a rapid application system identical to that previously described (Cuevas and Berg, 1998).

A DG-4 high-speed wavelength switcher (Sutter Instruments Co., Novato, Calif.) was used to apply alternating excitation light, and fluorescent emission was captured using a Sensicam digital CCD camera (Cooke Corporation, Auburn Hills, Mich.) and recorded with Slidebook 3.0 software (Intelligent Imaging Innovations, Denver, Colo.). Changes in [Ca²⁺]ᵢ were calculated using the Slidebook 3 software (Intelligent Imaging Innovations) using methods described previously (DeHaven and Cuevas, 2004).

In Vitro Ischemia

In vitro ischemia was achieved using the sodium azide/glucose deprivation model. This model for ischemic neuronal injury has been used effectively in numerous studies to mimic in vivo stroke in an in vitro environment, and has been shown to elicit electrophysiological and neurochemical changes that are qualitatively identical to the oxygen/glucose deprivation model of ischemia (Murai et al., 1997; Finley et al., 2004). The major advantage of the sodium azide/glucose deprivation model over the oxygen/glucose deprivation is that it elicits neurochemical responses that are significantly more rapid and robust (Finley et al., 2004), thus facilitating the recording of changes in [Ca²⁺]ᵢ.

Data Analysis

Analyses of these data were conducted using the SigmaPlot 2000 program (SPSS Science, Chicago, Ill.). Data points represent means±standard error of the mean (SEM). Statistical difference was determined using paired f-test for within-group experiments and unpaired f-test for between group experiments. For multiple group comparison an ANOVA was used followed by post-hoc analysis with a Dunn's. Differences were considered significant if p<0.05.

Solutions and Reagents

The control bath solution for all experiments was a physiological saline solution (PSS) containing (in mM) 140 NaCl, 1.2 MgCl₂, 3 KCl, 2.5 CaCl₂, 7.7 glucose and 10 HEPES, pH to 7.2 with NaOH. All drugs were applied in this solution unless otherwise noted. In vitro ischemia was induced by addition of the cytochrome oxidase inhibitor, NaN₃ (4 mM), and removal of glucose from the PSS. For experiments in which multiple ischemic episodes were induced in a single cell, the order of drug application was alternatively reversed to compensate for any effects due to rundown, desensitization or ischemic preconditioning. For experiments with metaphit, cells were preincubated in 50 μM metaphit during the last 15 mm of fura-2 loading and immediately prior to experiments being conducted. The metaphit was washed off for 5 min in the bath using PSS.

All chemicals used in this investigation were of analytic grade. The following drugs were used: tetrodotoxin (TTX), DTG, ibogaine and metaphit (Sigma-Aldrich, St. Louis, Mo.); BD-1047, carbetapentane, and PRE-084 (Tocris Bioscience, Ellisville, Mo.); ryanodine and thapsigargin (Alomone Labs, Jerusalem, Israel); and fura-2-AM (Molecular Probes, Eugene, Oreg.).

Animals

Fifty eight adult male Sprague-Dawley rats (Harlan, Indianapolis, Ind.) weighing 300 to 350 g were housed in a climate controlled room with water and laboratory chow available ad libidum. Animals were cared for according to the guidelines of the IACUC of the University of South Florida's College of Medicine.

Permanent Middle Cerebral Artery Occlusion

MCAO surgery was performed as previously reported by Vendrame et al (Vendrame et al., 2004) and originally described by Longa et al (Longa et al., 1989). Laser Doppler Radar (LDR) was used to monitor decrease in blood perfusion which indicates successful occlusion (Moor Instruments Ltd, Devon, England). A 2 mm diameter hole was drilled into the right parietal bone (1 mm posterior and 4 mm lateral from bregma), and a guide screw was set. The LDR probe (MP10M200ST; Moor) was inserted into the guide screw, and the tip of the probe was placed against the pial surface of the brain. Rats that did not show >55% reduction in perfusion during MCAO were excluded from the study because they generally failed to exhibit infarct damage. For MCAO, the embolus (4 cm long, 6 lb test monofilament) was advanced up the internal carotid artery into the middle cerebral artery and tied off at the internal/external carotid junction to produce permanent occlusion. The rat was then sutured, given a 1 ml subcutaneous injection of saline, and allowed to wake in a fresh cage.

Treatments and Tissue Preparation

Rats were randomly assigned to 1 of 7 groups: (1) MCAO (n=7); (2) MCAO and 15 mg/kg DTG in a 3% lactic acid vehicle (n=11); (3) MCAO and 30 mg/kg DTG (n=16); (4) MCAO and bi-daily injections of 30 mg/kg DTG (n=12); (5) MCAO and 10 mg/kg N-[2-(3,4-dichlorophenyl)ethyl]-N-methyl-2-(dimethylamino)ethylamine (BD 1047) (n=4); (6) MCAO and 10 mg/kg BD1047+30 mg/kg DTG (n=4); (7) sham/MCAO and 15 mg/kg DTG (n=4). DTG and BD1047 were obtained from Sigma Chemical Co (St. Louis, Mo.) and Tocris (Ellisville, Mo.), respectively. Injections were administered 24, 48, and 72 hours post MCAO. All rats received daily injections of 0.04 ml of ketophen and 1 ml of saline. The animals were sacrificed at 96 hours, and perfused with saline and 4% paraformaldehyde. The brains were harvested, fixed in paraformaldehyde, immersed in serial solutions of 20% and 30% sucrose, and sliced into 30 μm sections. Sections were either cold mounted on slides or placed in Walter's Anti-freeze cryopreservative.

Fluoro-Jade Histochemistry

Coronal brain sections from 1.7 to −3.3 mm from bregma containing cortical, striatal and hippocampal regions were stained with Fluoro-Jade. Fluoro-Jade labels degenerating neurons, and is more sensitive than triphenyltetrazolium chloride (TTC) for identifying neurodegeneration (Duckworth et al., 2005). This method was adapted from that originally described by Schmued et al.(Schmued et al., 1997) and has been detailed previously by Duckworth et al. (Duckworth et al., 2005). Tissue was cold mounted, thawed, and dried onto glass slides. Slides were sequentially placed in 100% ethanol for 3 min, and 70% ethanol and deionized water for 1 min each. Sections were then oxidized using 0.06% KMnO₄ solution for 15 min followed by three rinses for 1 minute each in PBS. Sections were stained in a 0.001% solution of Fluoro-Jade (Histochem, Jefferson, Ark.) in 0.1% acetic acid for 30 min. Slides were rinsed with PBS, allowed to dry at 45° C. for 20 min, cleared with xylene, and cover slipped were affixed to slides with DPX medium (Electron Microscopy Sciences, Ft. Washington, Pa.).

Immunohistochemistry and Histochemistry

Immunohistochemistry was performed as previously detailed by Butler et al(Butler et al., 2002). Free floating brain sections were pre-incubated in permeabilization buffer, 0.3% lysine, 0.3% TritonX-100, and 2% goat serum in phosphate buffered saline (PBS) for 30 min. Sections were washed three times with PBS between each incubation step. The tissue was then incubated in primary antibody solution overnight either with mouse anti-glial fibrillary acidic protein monoclonal antibody (GFAP) at a 1:10,000 dilution (MAB3402, Chemicon, Temecula, Calif.) or mouse anti-neuronal nuclei monoclonal antibody (NeuN) at a 1:30,000 dilution. Sections exposed to antibodies directed against these proteins were subsequently incubated for 1 hour in biotinylated horse anti-mouse secondary antibody solution (Vector Laboratories, Burlingame, Calif.) followed by avidin/biotin/horseradish peroxidase complex (VectastainElite ABC kit; Vector) for 1 h. Sections were then washed 3× in PBS, and metal-enhanced 3,3′-diaminobenzidine (Pierce, Rockford, Ill.) was used to for color development. For experiments involving Griffonia simplicifolia Isolectin (IB4/Alexa Fluor 488; Molecular Probes Eugene, Oreg.), sections were incubated overnight in isolectin IB4 diluted 1:1000. All sections were mounted on slides, dried, cleared sequentially with 100%, 95%, and 70% ethanol and xylene, and coverslips affixed with DPX.

Infarct Area and immunohistochemistry Quantification

Images of Fluoro-Jade stained brain sections, 5 per corticalustriatal and 4 per cortical/hippocampal regions for each rat from 1.7 to −3.3 mm from bregma, were acquired with the Olympus IX71 microscope controlled by DP manager software (Olympus America Inc, Melville, N.Y.) at a magnification of 12.5×. All other images were taken with a Zeiss Axicam Color (model 412-312) camera and Zeiss Axioscope 2 (model 801572) microscope controlled by Openlab software (Improvision Ltd, Lexington Mass.). Images were edited with Jasc Paintshop Pro to sharpen and enhance contrast of the images to the same specifications. Image analysis was performed utilizing NIH Image J software to determine the area of neurodegeneration by particle analysis in the regions of interest. The area of the contralateral side of the brain tissue was measured and used to compensate for possible edema in ipsilateral hemispheres. NeuN immunostaining was analyzed using the NIH Image J software to count NeuN positive cells by particle analysis in the ipsilateral hemispheres of the corticalustriatal and corticaluhippocampal regions of the rat brains at a magnification of 10×.

Statistical Analysis

Data were analyzed using SigmaPlot 2000 (SPSS Science, Chicago, Ill.). Data points represent means±standard error of the mean (SEM). Multiple group comparisons were conducted using a One-Way or a Two-Way ANOVA, as appropriate, followed by post-hoc analysis with a Tukey or Dunn's Test to identify differences between individual groups. Differences were considered significant if p<0.05. Survival rates were analyzed using a Kaplan-Meier Survival Analysis and post-hoc with a Holm-Sidak test for pairwise multiple comparison.

Cell Culture

Cortical neurons from embryonic (e18) rats were used to study the effects of sigma receptor activation on ASIC function. Mothers were sacrificed by decapitation. Once the uterus was removed, the embryos were dissected out, the cortex was removed and minced, and placed in an isotonic buffer solution containing (in mM): 137 NaCl, 5 KCl, 0.2 NaH2PO4, 0.2 KH2PO4, 5.5 glucose, 14.8 sucrose, and titrated to pH 7.4 with NaOH. The tissue was digested in the isotonic buffer containing 0.25% trypsin/EDTA and incubated for 10 minutes at 37° C. Dissociated neurons were then diluted in 3× volume with Dulbecco's Modified Eagle Media containing 10% fetal bovine serum and 100 U/ml penicillin and 0.1 mg/ml streptomycin. Cells were counted using a hemocytometer, plated (0.5×10⁶ cells) on 18 mm pre-treated poly-l-lysine coverslips, and incubated at 37° C. under 95% air, 5% CO2 atmosphere. After a 24 hour incubation, the media was replaced with Neurobasal medium supplemented with B27 and 0.5 mM L-glutamine (NB medium). Cells were used for studies after 14-21 days in culture.

Ca2+ Imaging

Prior to recordings, the coverslip was incubated for 1 hour at 22° C. in physiological saline solution (PSS) containing (in mM): 140 NaCl, 5.4 KCl, 25 HEPES, 20 glucose, 1.3 CaCl2 and 1.0 MgCl2 titrated to pH 7.4 with NaOH and 1 μM Fura-2-AM. The coverslip was then washed 3 times with PSS (fura-free). All drugs were applied in PSS, and ASIC channels were activated by applying PSS with a pH of 6.0 (± drug). Spontaneous activity was suppressed using 500 nM TTX. The solutions were rapidly applied using an 8-barrel applicator controlled by a Piezo-Electric Transducer (Piezo Systems Inc., Cambridge, Mass.). Single-cell ratiometric Ca2+ fluorometry was conducted using a Lambda DG-4 (Sutter Instrument Co., Novato, Calif.) for illumination (340 nM and 380 nM, wavelength), and emission fluorescence at 510 nm was captured using a Cooke Sensicam digital CCD camera (Cooke Co., Auburn Hills, Mich.) and SlideBook software (Intelligent Imaging Innovations, Denver, Colo.).

Results

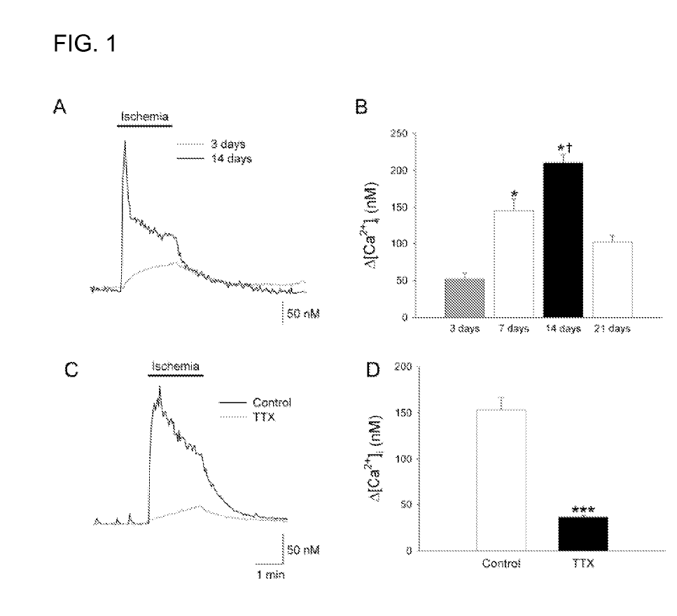

Experiments were conducted to characterize the changes in intracellular calcium evoked by the sodium-azide/glucose deprivation model of in vitro ischemia. FIG. 1A shows representative traces of change in intracellular Ca²⁺ as a function of time evoked by rapid induction of chemical ischemia in two cortical neurons that had remained in culture for 3 or 14 days, respectively. Following 3-4 days in culture, chemical ischemia elicited small, slow rising elevations in [Ca²⁻]ᵢ in the neurons, whereas after 7 days in culture, ischemia evoked rapid increases in [Ca²⁺]ᵢ. The increases in [Ca²⁺]ᵢ observed at later time points (7-21 days in culture) in response to ischemia were transient, and [Ca²⁺]ᵢ returned to control levels in >80% of the cells tested following washout with control PSS (n>1000). A plot of mean peak change in [Ca²⁺]ᵢ shows that the response to chemical ischemia increased significantly from 3 to 14 days in culture, and the elevations in [Ca²⁺]ᵢ diminished when the neurons were in culture for over 14 days (FIG. 1B).

Previous studies have shown that chemical ischemia promotes the release of excitatory neurotransmitters which may elicit these elevations in [Ca²⁺]ᵢ (Djali and Dawson, 2001). Therefore, experiments were conducted to determine if inhibition of voltage-activated Na⁺ channels, and consequently neurotransmission, with TTX (200 nM) abolished the elevations in [Ca²⁺]ᵢ induced by chemical ischemia. FIG. 1C shows representative [Ca²⁺]ᵢ traces of responses evoked by ischemia in the absence and presence of TTX. Inhibition of synaptic transmission with TTX depressed ischemia-evoked increases in [Ca²⁺]ᵢ relative to control. A plot of maximal increase in [Ca²⁺]ᵢ in the absence and presence of TTX is shown in FIG. 1D. The ischemia-evoked increase in [Ca²⁺]ᵢ was decreased in a significant manner by 65±4% in the presence of TTX.

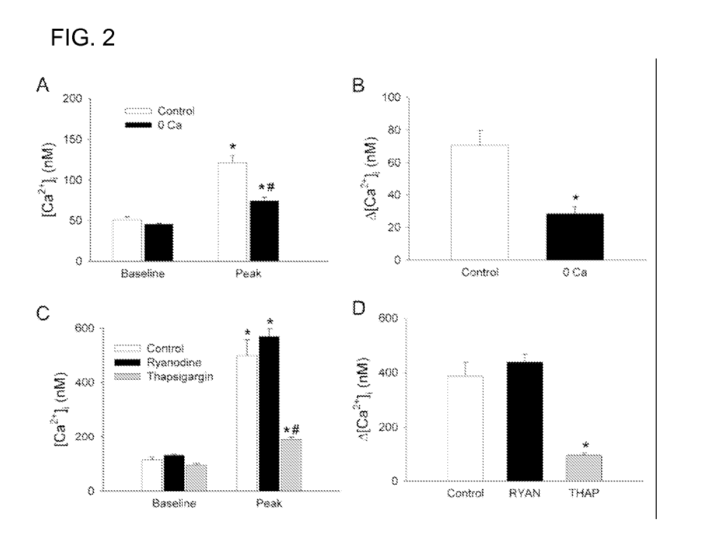

Further experiments were conducted to resolve the source of calcium mediating the elevations in [Ca²⁺]ᵢ observed in response to chemical ischemia. To determine if extracellular calcium contributed to the increase in [Ca²⁺]ᵢ ischemic conditions were induced in the absence and presence of extracellular calcium. Under both conditions, elevations in [Ca²⁺]ᵢ were noted (FIG. 2A), but in the absence of extracellular calcium, the peak increases in [Ca²⁺]ᵢ were significantly less than those observed in control experiments. Thus, a component of the ischemia-induced increase in [Ca²⁺]ᵢ depends on the presence of extracellular calcium. Given that elimination of extracellular calcium did not abolish the increase in [Ca²⁻]ᵢ, we investigated the role of calcium release from intracellular stores in the response to ischemia. Ryanodine (10 μM) was used to selectively inhibit release from caffeine/ryanodine-sensitive calcium stores; whereas, the sarcoplasmic/endoplasmic reticulum Ca²⁺-ATPase inhibitor, thapsigargin (10 μM), was used to depleted both ryanodine- and IP3-sensitive stores. Ischemia-increases in [Ca²⁺]ᵢ were observed in control, ryanodine, and thapsigargin experiments (FIG. 2B). However, preincubation in thapsigargin decreased the peak elevation in [Ca²⁺]ᵢ, whereas ryanodine did not significantly alter the effects of ischemia on [Ca^{2|}]ᵢ (FIG. 2B). Taken together these data suggest that elevations of [Ca²⁺]ᵢ observed in response to chemical ischemia are in part due to calcium release from IP3-sensitive stores, but do not appear to involve liberation of calcium from ryanodine sensitive stores.

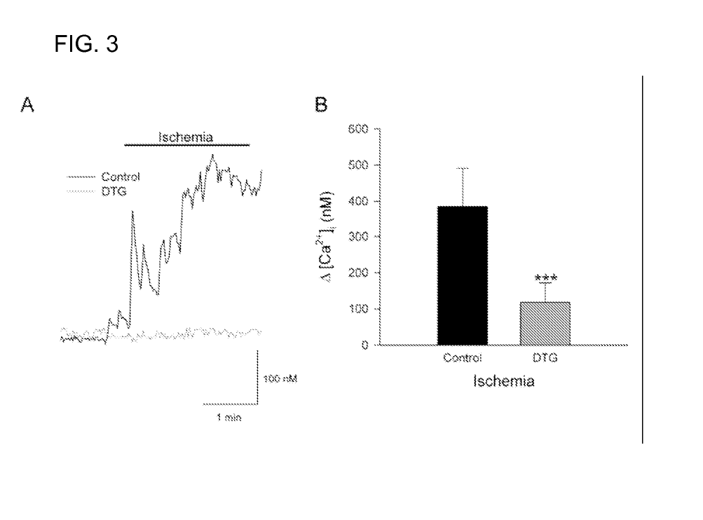

Experiments were carried out to determine if stimulation of sigma receptors modulates the changes in [Ca²⁺]ᵢ evoked by in vitro ischemia. FIG. 3A shows representative traces of [Ca²⁺]ᵢ recorded from a single neuron in response to ischemia in the absence (Control) and presence of 50 μM DTG (DTG). The elevation in [Ca²⁺]ᵢ evoked by ischemia was abolished when the sigma receptor ligand was coapplied. A bar graph of ischemia-induced mean peak increase in [Ca²⁺]ᵢ observed in 13 neurons in the absence and presence of 50 μM DTG is shown in FIG. 3B, and demonstrates that DTG decreases the rise in [Ca²⁺]ᵢ by—70%. This effect of DTG was statistically significant and was reversible upon washout of the sigma agonist (data not shown).

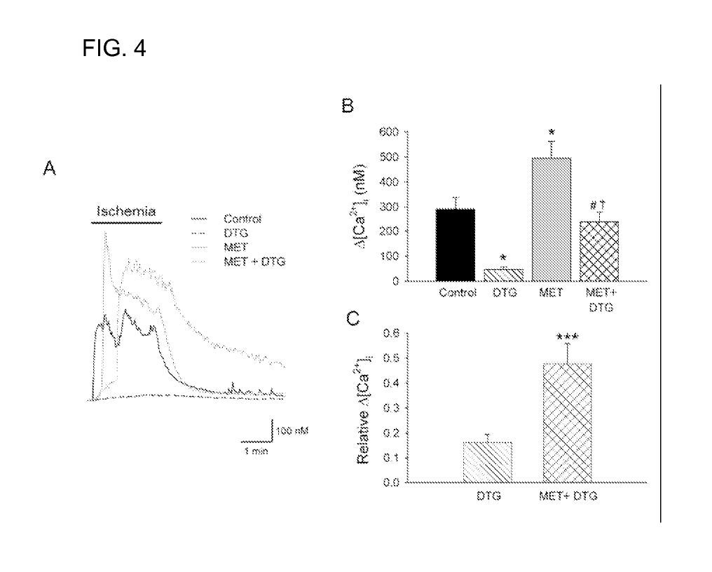

To confirm that the effects of DTG on [Ca²⁺]ᵢ are mediated via the activation of sigma receptors, the sigma receptor antagonist, metaphit, was used in a series of experiments. Cells were exposed to ischemia in the absence and presence of 50 .tM DTG, with or without preincubation in metaphit (50 μM). Whereas DTG depressed the ischemia induced elevation in [Ca²⁺]ᵢ in control cells, the responses observed in cells pretreated with metaphit were comparable in the absence and presence of DTG. Moreover, both responses (±DTG) observed in metaphit pretreated cells were larger than the control response (no metaphit pretreatment). In similar experiments, cells not exposed to metaphit responded to DTG with a decrease in ischemia-induced elevations in [Ca²⁺]ᵢ from a control value of 291±47 nM to 47±9 nM in the presence of the sigma agonist (FIG. 4B). Cells preincubated in metaphit displayed a more robust increase in [Ca²⁺]ᵢ during ischemia (495±68 nM) relative to control cells (FIG. 4B). Furthermore, neurons pretreated with metaphit continued to exhibit pronounced elevations in [Ca²⁺]ᵢ in the presence of DTG (237±39 nM). These increases in [Ca²⁺]ᵢ were significantly greater (p<0.01) than those observed in control neurons exposed to DTG and were comparable to those seen in control neurons not exposed to DTG. To confirm that the difference observed in responses to DTG and DTG following metaphit preincubation were not the result of metaphit augmentation of the [Ca²⁻]ᵢ responses, the responses were normalized to the mean of their respective controls. FIG. 4C shows a bar graph of the relative change in [Ca²⁺]ᵢ observed in the presence of DTG in control neurons (DTG) and neurons preincubated in metaphit (MET+DTG). Whereas DTG decreased the elevation in [Ca²⁺]ᵢ evoked by ischemia in control cells by 83±3%, the sigma receptor agonist only reduced the response by 52±8% in cells preincubated in the irreversible sigma receptor antagonist.

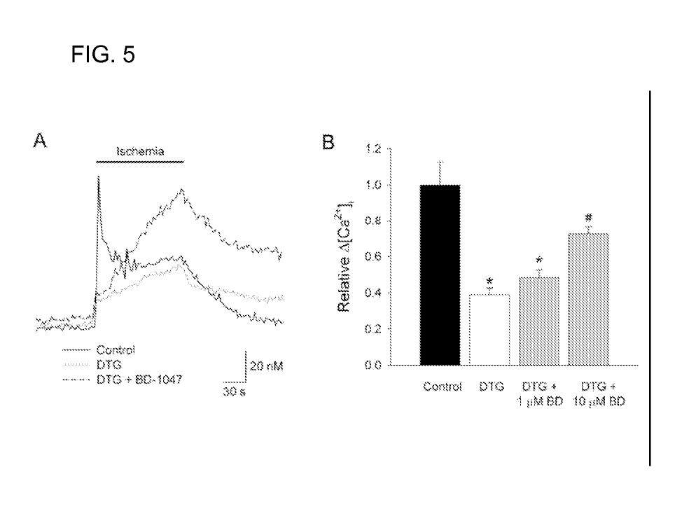

A second sigma receptor-selective antagonist, BD-1047 (Matsumoto et al., 1995), was used to further support that DTG was acting via the stimulation of sigma receptors. FIG. 5A shows intracellular calcium traces obtained from three neurons in response to chemical ischemia in the absence (Control) and presence of 10 μM DTG (DTG) or 10 μM DTG following a 5 min preincubation in 10 μM BD-1047 (DTG+BD-1047). While DTG reduced the ischemia elicited elevations in [Ca²⁺]ᵢ, application of BD-1047 diminished the effectiveness of DTG. In similar experiments, the effects of DTG on ischemia-evoked calcium transients were blocked by 1 pM and 10 pM BD-1047 in a concentration-dependent and statistically significant manner (FIG. 5B). These two concentrations of BD-1047 reduced the effects of DTG by 15% and 55%, respectively.

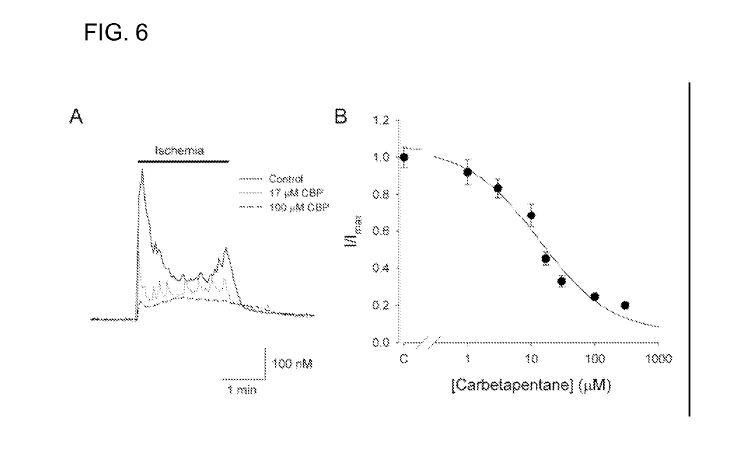

DTG and metaphit are pan-selective sigma ligands, acting on both sigma-1 and sigma-2 receptors, and the concentrations of BD-1047 used here cannot definitively discriminate between the receptor subtypes. Therefore, experiments were conducted using sigma receptor subtype-selective agonists to determine the specific sigma receptor subtype(s) responsible for the depression of ischemia induced increases in [Ca²⁺]ᵢ FIG. 6A shows representative traces of [Ca²⁻]ᵢ recorded from three neurons in the absence (Control) and presence of the sigma-1 selective agonist, carbetapentane, at the indicated concentrations. Carbetapentane reduced the effect of ischemia on [Ca²⁺]ᵢ in a concentration dependent manner, and this effect of carbetapentane was reversible upon washout of drug (data not shown). FIG. 6B shows a plot of the relative ischemia-induced increases in [Ca²⁺]ᵢ as a function of carbetapentane concentration. A fit of the data using a Langmuir-Hill equation indicated that the sigma-1 selective ligand inhibits the effects of ischemia on [Ca²⁺]ᵢ with a half-maximal concentration of 13.3 μM and with a Hill Coefficient of 0.8.

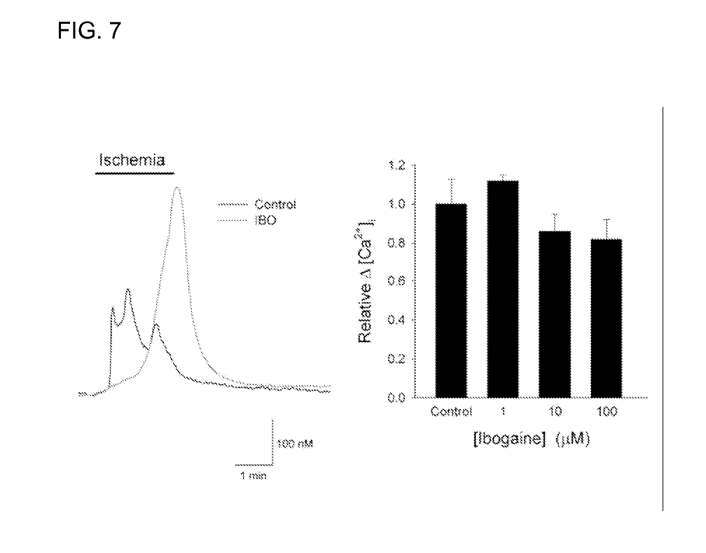

Additional experiments were conducted to determine if sigma-2 receptors contribute to the DTG-mediated inhibition of ischemia-induced increases in [Ca²⁺]ᵢ. For these experiments the sigma-2 receptor-selective agonist, ibogaine, was used. FIG. 7A shows representative traces of [Ca²⁺]ᵢ recorded in response to ischemia in the absence and presence of 100 μM ibogaine. Unlike carbetapentane, ibogaine failed to inhibit the ischemia-induced elevations in [Ca²⁻]ᵢ. In similar experiments, ibogaine at a concentration range of 1-100 μM, which has been shown to block sigma-2 mediated events (Zhang and Cuevas, 2002), failed to inhibit the effects of ischemia on [Ca²⁺]ᵢ (FIG. 7B). This observation suggests that the sigma-1 receptor is primarily responsible for the depression of ischemia-induced increase in [Ca²⁺]ᵢ mediated by DTG.

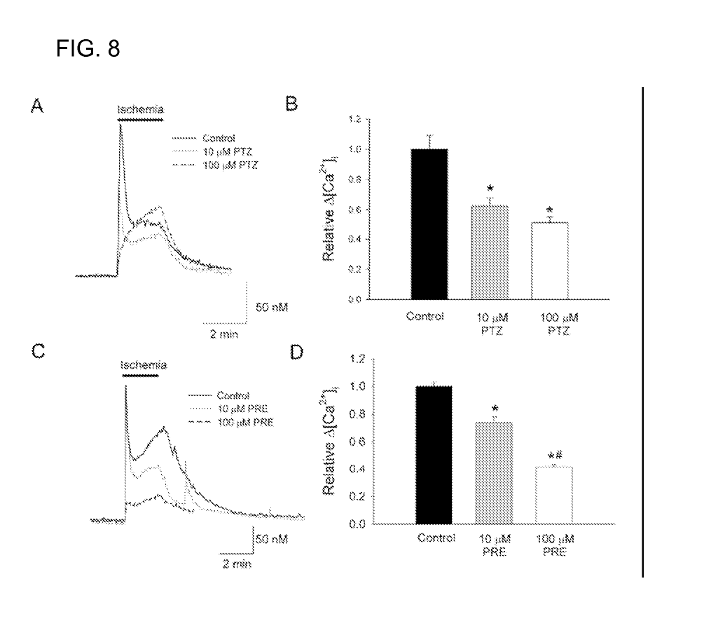

To confirm that sigma-1 activation attenuates ischemia-induced increase in [Ca^{2|}]ᵢ mediated by DTG and carbetapentane, neurons were treated with the sigma-1 selective agonists (+)-pentazocine and PRE-084. FIG. 8A shows representative traces of [Ca^{2|}]ᵢ recorded during ischemia from 3 neurons in the absence (Control) and presence of (+)-pentazocine at the indicated concentrations. Peak elevations in [Ca²⁺]ᵢ were significantly depressed by (+)-pentazocine in a concentration dependent manner. Application of 10 μM (+)-pentazocine decreased the ischemia-induced elevations in [Ca²⁺]ᵢ by 37±5%, whereas 100 μM (+)-pentazocine depressed the change in [Ca²⁺]ᵢ by 49±4% (FIG. 8B). Responses to ischemia were also blocked by application of PRE-084 (FIG. 8C). Both 10 μM and 100 μM PRE-084 decreased elevations in [Ca²⁺]ᵢ in a statistically significant manner. These decreases were 26±4% and 58±1%, respectively (FIG. 8D).

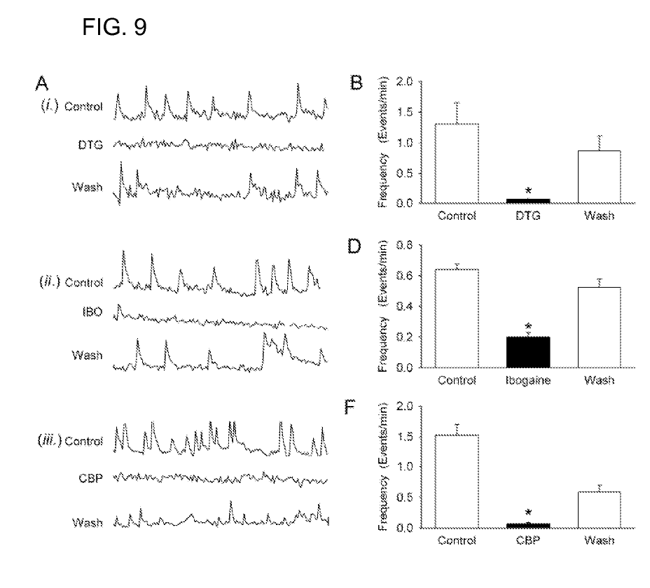

Spontaneous elevations in [Ca²⁺]ᵢ were frequently observed in our experiments (see FIG. 8C, Control trace). These elevations in [Ca²⁺]ᵢ nearly always occurred in multiple neurons in the same visual field in a synchronized manner (data not shown). While our data demonstrate that activation of sigma receptors decreased the ischemia-induced elevations of [Ca²⁺]ᵢ, further experiments were conducted to determine if spontaneous increases in [Ca²⁺]ᵢ were also modulated by sigma receptors. FIG. 9A shows traces of spontaneous activity recorded from a single cortical neuron in the absence (Control) and presence of 100 μM DTG (i.) and following washout of drug (Wash). DTG was found to reversibly block spontaneous increases in [Ca²⁺]ᵢ in a statistically significant manner (FIG. 9B). To identify the subtype of sigma receptor involved in the modulation of spontaneous calcium transients, the sigma-2-selective agonist, ibogaine, was used. Traces of spontaneous activity recorded from a single cortical neuron in the absence (Control, Wash) and presence of 50 μM ibogaine (IBO) are shown in FIG. 9A, ii. In identical experiments, bath application of the sigma-2 agonist significantly decreased the number of spontaneous calcium events (FIG. 9C). Activation of sigma-1 receptors with carbetapentane (100 μM) also affected spontaneous activity (FIG. 9A, iii), resulting in a significant decrease in the number spontaneous of Ca transients observed in the cells (FIG. 9D).

DTG Dose Quantification

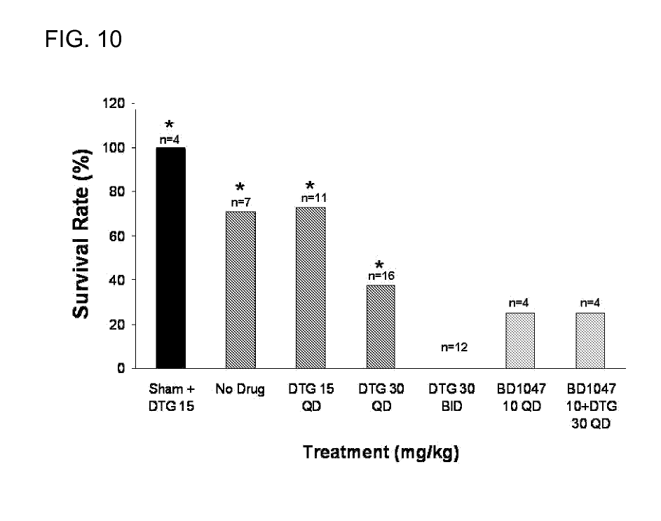

Reports in the literature indicate that doses of DTG as high as 30 mg/kg are well tolerated by rats (Rawls et al., 2002). Thus, this high dose of DTG was used to determine if stimulation of sigma receptors was neuroprotective at delayed time points. Furthermore, to confirm that the effects of DTG were mediated by activation of sigma receptors, the sigma receptor selective antagonist, BD1047, was used at concentrations (10 mg/kg) previously shown to abolish systemic effects of DTG (Rawls et al., 2002). Rats received subcutaneous injections of DTG at 30 mg/kg daily, 15 mg/kg daily, 30 mg/kg bi-daily, 10 mg/kg daily BD1047, or BD1047 (10 mg/kg)+DTG (30 mg/kg) administered at 24, 48 and 72 hours post surgery with animals being sacrificed at 96 hours. The percent survival was defined as the number of rats that survived 96 hours post MCAO divided by the total number of rats that awoke from anesthesia. The MCAO and 15 mg/kg daily groups produced survival rates of over 70%, while 30 mg/kg daily had a survival rate under 40% (FIG. 10). In contrast, the survival rates of BD1047 alone, BD 1047+DTG, and 30 mg/kg bi-daily were <25%, indicating that high concentrations of DTG and inhibition of sigma receptors worsen survival outcomes. Given that none of the sham animals died following DTG application, high concentrations of DTG are not lethal in the absence of MCAO (FIG. 10). The dose of 15 mg/kg per day did not enhance mortality relative to the MCAO only group, and therefore was the dose chosen for the subsequent study.

DTG treatment 24-hr Post-MCAO Decreases Infarct Size

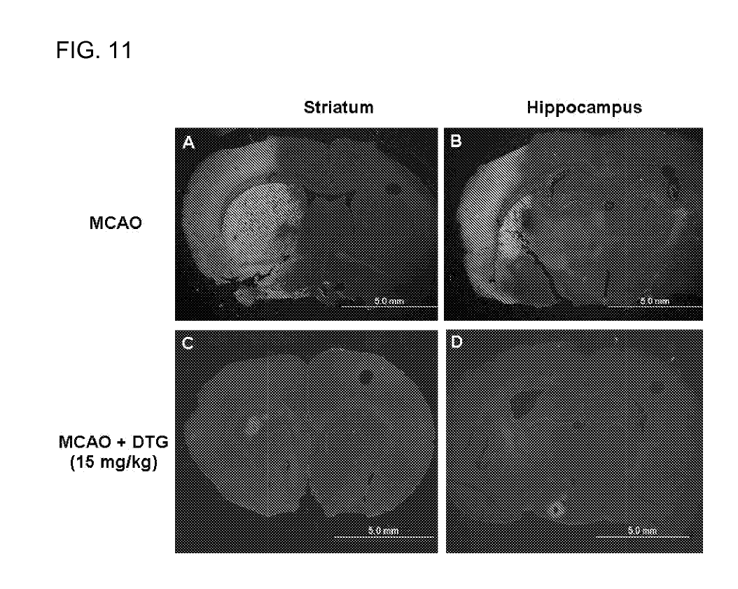

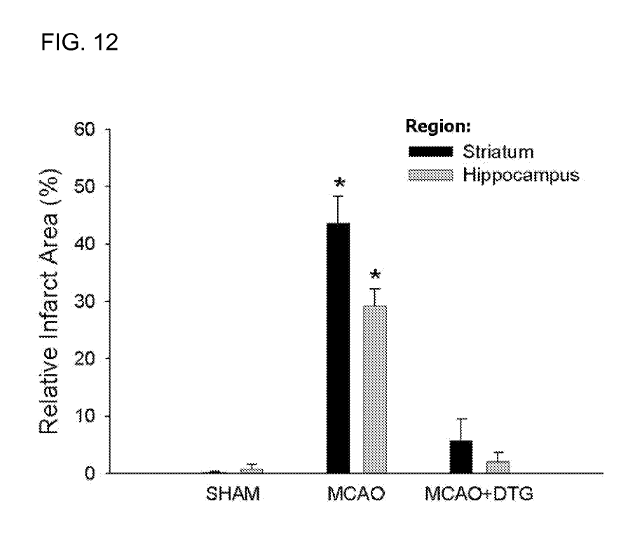

The marker for neurodegeneration, Fluoro-Jade, was used to determine infarct area after MCAO. FIG. 11 shows representative photomicrographs of coronal sections from cortical/striatal (FIGS. 11A and 11C) and cortical/hippocampal (FIGS. 11B and 11D) regions obtained form rats in the absence and presence of DTG treatment post-MCAO. Fluoro-Jade staining was observed in brain sections from all animals that underwent MCAO, but absent from sections collected from sham controls. While Fluoro-Jade staining was observed in DTG treated (15 mg/kg) animals (FIG. 11C-D), this was less pronounced than in MCAO only rats (FIG. 11A-B). Analysis of similar sections obtained from the individual groups showed that Fluoro-Jade labeled 44±5% and 29±3% of the cortical/striatal and cortical/hippocampal regions, respectively, in MCAO-only rats. However, in DTG-treated animals (n=7) infarct areas were 6±4% and 2±2% in the cortical/striatal and cortical/hippocampal regions, respectively (FIG. 12). This decrease in infarct area was statistically significant (p<0.001), and demonstrates that DTG application at delayed time points can effectively decrease stroke-induced neurodegeneration.

DTG-induced Enhancement of Neurosurvival

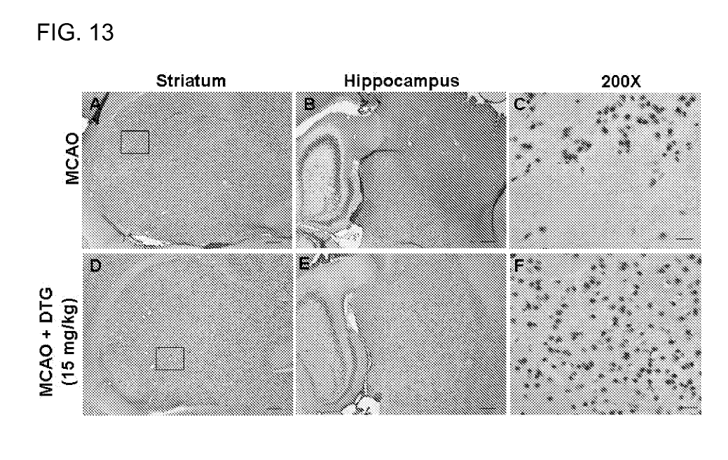

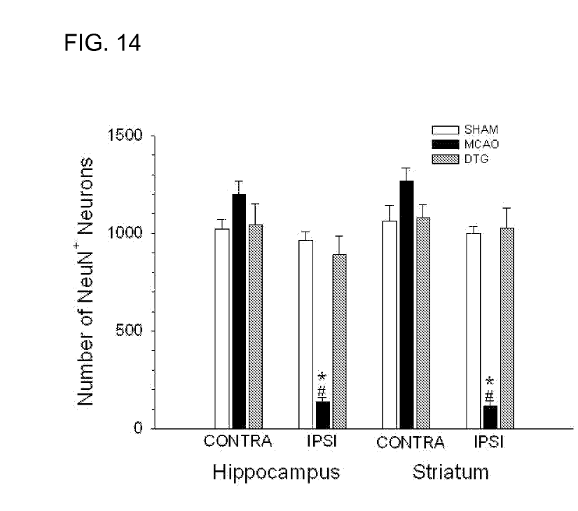

To confirm that DTG increase the number of neurons surviving MCAO in rats, we immunostained for the neuron-specific protein, NeuN, to selectively label the nuclei of viable cells. FIG. 13 shows representative brain sections from DTG treated and untreated rats immunostained for NeuN. Whereas NeuN expression was absent from the infarct zone of MCAO-only rats (FIG. 13A-C), NeuN staining was readily visible in the infarct zone of animals injected with DTG (FIG. 13D-F). In identical experiments, the total number of NeuN-positive neurons were counted by image analysis, and average number per visual field determined. These values are shown in FIG. 14, and demonstrate that MCAO significantly decreases the number of viable neurons in the ipsilateral cortical/striatal and cortical/hippocampal infarct zones, relative to the respective regions in the contralateral hemisphere. Injection with DTG significantly increased the number of viable neurons in both ipsilateral cortical/striatal and cortical/hippocampal infarct zones (p<0.001). Furthermore, the number of neurons detected in these areas of the ipsilateral hemisphere was comparable to those observed in equivalent areas of the contralateral side (FIG. 14). Thus, DTG increases neuronal survival to the extent that the number of surviving cells was not statistically different from that observed in sham controls (FIG. 14).

DTG Treatment Decreases Expression of Inflammatory Markers

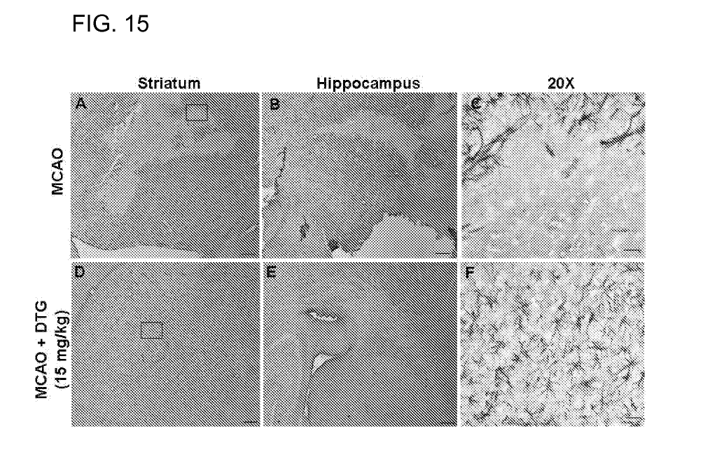

It has been shown that reduced brain inflammation is a key component to treating stroke at delayed time points (Newcomb et al., 2005; Vendrame M et al., 2005). Thus, to ascertain if stimulation of sigma receptors by DTG exerts an anti-inflammatory response, we examined inflammatory markers for both astrocytes and microglia in brain sections from the experiments discussed above. Reactive astrocytes, which participate in the inflammatory response in the brain following injury (O'Callaghan, 1994), exhibit high levels of the distinguishing marker, GFAP. Thus, GFAP immunoreactivity was used to label reactive astrocytes responding to the ischemic injury produced by our model. Representative images showing GFAP labeling in tissue sections collected from MCAO-only and DTG treated animals are shown in FIG. 15. Astrocytes containing high levels of GFAP were always observed in MCAO-only animals, and these astrocytes were primarily located in the areas surrounding the infarction (FIG. 15A-C). However, the area inside of the infarction was noticeably devoid of astrocytes expressing GFAP in these sections. In animals receiving DTG, brain sections exhibited a marked decrease in the level of GFAP expression. Moreover, astrocytes with this low level GFAP-labeling were detected throughout the infarct zone (FIG. 15D-F).

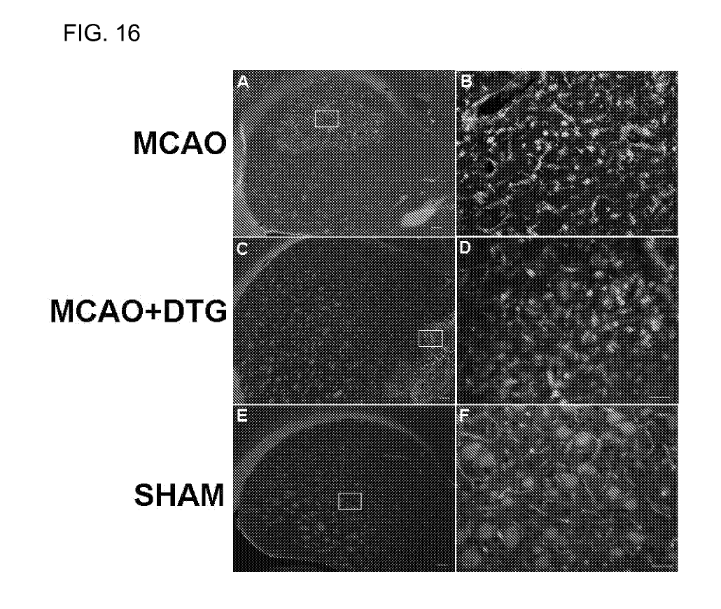

Inflammatory response of the central nervous system also involves activation of microglia and infiltration of systemic macrophages. Both of these cells express the surface protein IB4 when activated in response to injury, and can be selectively labeled in this state using isolectin IB4 (Goldstein and Winter, 1999). FIG. 16 shows representative photomicrographs of tissue sections of corticalustriatal and cortical/hippocampal regions labeled with isolectin IB4 from animals subjected to MCAO with and without DTG treatment. MCAO evokes a pronounced increase in isolectin IB4 labeled cells in the infarct zone of untreated animals (FIG. 16A-B). However, MCAO fails to elicit these elevations in isolectin IB4-positive cells in the infarct zone of sections taken from animals treated with DTG (FIG. 16C-D). Isolectin IB4 labeling was also absent from sections taken from sham control animals (FIG. 16E-F). Taken together, our data suggest that application of DTG blunts the inflammatory response of the brain following MCAO. This depression of neuroinflammation is likely to contribute to the enhanced neuronal survival reported here.

Stroke is the third leading cause of death in the industrialized world, and a major cause of long-term disability. Ischemic stroke results from cerebral artery occlusion leading to restricted blood flow to the brain, and triggers a series of events that ultimately evoke neuronal death in the affected and surrounding areas. Brain ischemia is associated with glucose-oxygen deprivation and a cellular switch to anaerobic glycolysis. The accumulation of lactic acid produced by anaerobic glycolysis leads to acidosis in the ischemic region. This acidosis contributes to the demise of neurons.

One of the consequences of acidosis is the activation of acid-sensing ion channels (ASICs). ASICs compose a family of non-selective cation channels that are expressed in both peripheral and central nervous system neurons, and are activated by extracellular protons. These channels are involved in various processes such as nociception and mechanoreception. In central nervous system, ASICs are involved in synaptic plasticity, learning and memory. Moreover, ASICs have a high permeability to calcium and have been implicated in neuronal death following ischemic brain injury. Transgenic mice deficient in ASIC1 have reduced infarct size and volume in response to middle cerebral artery occlusion relative to wild-type mice. The increases in calcium evoked by acidosis were also absent in transgenic mice neurons lacking ASIC1, giving insight into how these channels may contribute to neuronal injury. Thus, ASICs are a putative target for neuroprotection following an ischemia insult.

Our laboratory has shown that activation of sigma receptors inhibits multiple membrane ion channel subtypes in neurons, and that stimulation of these receptors is neuroprotective following ischemic injury. Thus, experiments were conducted to determine if sigma receptor activation regulates ASIC function in cortical neurons. These experiments were designed to give insight into a putative mechanism of sigma receptor-mediated neuroprotection.

### REFERENCES

Ajmo, C. T., Vernon, D. O., Collier, L., Pennypacker, K R., Cuevas, J. Sigma receptor activation reduces infarct size at 24 hours after permanent middle cerebral artery occlusion in rats. Current Neurovasc. Res. (In press)

Bowen W D (2000) Sigma receptors: recent advances and new clinical potentials. Pharm Acta HeIv 74:211-218.

Butler, T. L., Kassed, C. A., Sanberg, P. R., Willing, A. E., and Pennypacker, K. R.: Neurodegeneration in the rat hippocampus and striatum after middle cerebral artery occlusion. Brain Res 929 (2): 252-60, 2002.

Casellas P, Galiegue S, Bourrie 8, Ferrini J B, Jbilo 0 and Vidal H (2004) SR31747A: a peripheral sigma ligand with potent antitumor activities. Anticancer Drugs 15:113-118.

Choi, D. W. (1988a). Calcium-mediated neurotoxicity: relationship to specific channel types and role in ischemic damage. Trends Neurosci. 11. 465-469.

Choi, D. W. (1995). Calcium: still center-stage in hypoxic-ischemic neuronal death. Trends Neurosci. 18. 58-60.

Chu, X. P., Miesch, J., Johnson, M., Root, L., Zhu, X. M., Chen, D., Simon, R. P., and Xiong, Z. G. (2002). Proton-gated channels in PC12 cells. J. Neurophysiology. 87. 2555-2561.

Cuevas J and Berg D K (1998) Mammalian nicotinic receptors with alpha7 subunits that slowly desensitize and rapidly recover from alpha-bungarotoxin blockade. J Neurosci 18:10335-10344.

DeHaven W I and Cuevas J (2004) VPAC receptor modulation of neuroexcitability in intracardiac neurons: dependence on intracellular calcium mobilization and synergistic enhancement by PACI receptor activation. J Biol Chem 279:40609-40621.

Duckworth, E. A., Butler, T. L., De Mesquita, D., Collier, S. N., Collier, L., and Pennypacker, K. R.: Temporary focal ischemia in the mouse: technical aspects and patterns of Fluoro-Jade evident neurodegeneration. Brain Res 1042 (1): 29-36, 2005.

Ebine Y, Fujiwara N and Shimoji K (1994) Mild acidosis inhibits the rise in intracellular Ca2+ concentration in response to oxygen-glucose deprivation in rat hippocampal slices. Neurosci Left 168:155-158.

Finley M, Fairman D, Liu D, Li P, Wood A and Cho S (2004) Functional validation of adult hippocampal organotypic cultures as an in vitro model of brain injury. Brain Res 1001:125-132.

Hanner M, Moebius F F, Flandorfer A, Knaus H G, Striessnig J, Kempner E and Glossmann H (1996) Purification, molecular cloning, and expression of the mammalian sigmal-binding site. Proc. Natl. Acad. Sci. U.S.A 93: 8072-8077.

Hayashi T, Kagaya A, Takebayashi M, Shimizu M, Uchitomi Y, Motohashi N and Yamawaki S (1995) Modulation by sigma ligands of intracellular free Cai-+ mobilization by N-methyl D-aspartate in primary culture of rat frontal cortical neurons. J Pharmacol Exp Ther 275:207-214.

Hayashi T, Maurice T and Su T P (2000) Ca(2+) signaling via sigma(1)-receptors: novel regulatory mechanism affecting intracellular Ca(2+) concentration. J Pharmacol Exp Ther 293:788-798.

Hayashi T and Su T P (2003) Sigma-1 receptors (sigma(I) binding sites) form raft-like microdomains and target lipid droplets on the endoplasmic reticulum: roles in endoplasmic reticulum lipid compartmentalization and export. J Pharmacol Exp Ther 306:718-725.

Hayashi T and Su T P (2004) Sigma-1 receptor ligands: potential in the treatment of neuropsychiatric disorders. CNS Drugs 18 :269-284.

Hiramatsu M, Mizuno N and Kanematsu K (2006) Pharmacological characterization of the ameliorating effect on learning and memory impairment and antinociceptive effect of KT95 in mice. Behav Brain Res 167:219-225.

Hirata M, Mon T, Soga S, Umeda T, Ohmomo Y. (2006) Synthesis and in vitro evaluation of iodinated derivatives of piperazine as a new ligand for sigma receptor imaging by single photon emission computed tomography. Chem Pharm Bull 54:470-5.

Klette K L, DeCoster M A, Moreton J E and Tortella F C (1995) Role of calcium in sigma-mediated neuroprotection in rat primary cortical neurons. Brain Res 704:31-41.

Klette K L, Lin Y, Clapp L E, DeCoster M A, Moreton J E and Tortella F C (1997) Neuroprotective sigma ligands attenuate NMDA and trans-ACPD-induced calcium signaling in rat primary neurons. Brain Res 756:231-240.

Kume T, Nishikawa H, Taguchi R, Hashino A, Katsuki H, Kaneko S, Minami M, Satoh M and Akaike A (2002) Antagonism of NMDA receptors by sigma receptor ligands attenuates chemical ischemia-induced neuronal death in vitro. Eur J Pharmacol 455:91-100.

Lobner D and Lipton P (1990) Sigma-ligands and non-competitive NMDA antagonists inhibit glutamate release during cerebral ischemia. Neurosci Left 117:169-174.

Lockhart B P, Soulard P, Benicourt C, Privat A and Junien J L (1995) Distinct neuroprotective profiles for sigma ligands against N-methyl-D-aspartate (NMDA), and hypoxia-mediated neurotoxicity in neuronal culture toxicity studies. Brain Res 675:110-120.

Matsumoto R R, Bowen W D, Tom M A, Vo V N, Truong D D and De Costa B R (1995) Characterization of two novel sigma receptor ligands: antidystonic effects in rats suggest sigma receptor antagonism. Eur J Pharmacol 280:301-310.

Matsumoto R R, Hemstreet M K, Lai N L, Thurkauf A, De Costa B R, Rice K C, Hellewell S B, Bowen W D and Walker J M (1990) Drug specificity of pharmacological dystonia. Pharmacol Biochem Behav 36:151-155.

Mattson M P (2000) Apoptosis in neurodegenerative disorders. Nat Rev Mo! Cell Biol 1:120-129.

McCann D J, Weissman A D and Su T P (1994) Sigma-1 and sigma-2 sites in rat brain: comparison of regional, ontogenetic, and subcellular patterns. Synapse 17:182-189.

McCracken K A, Bowen W D and Matsumoto R R (1999) Novel sigma receptor ligands attenuate the locomotor stimulatory effects of cocaine. Eur J Pharmacol 365:35 38.

Monnet F P (2005) Sigma-1 receptor as regulator of neuronal intracellular Ca2+: clinical and therapeutic relevance. Biol Cell 97:873-883.

Monnet F P, Morin-Suwn M P, Leger J and Combettes L (2003) Protein kinase C-dependent potentiation of intracellular calcium influx by sigmal receptor agonists in rat hippocampal neurons. J Pharmacol Exp Ther 307:705-712.

Murai Y, Ishibashi H, Koyama S and Akaike N (1997) Ca2+-activated K+ currents in rat locus coeruleus neurons induced by experimental ischemia, anoxia, and hypoglycemia. J Neurophysiol 78:2674-2681.

Newcomb, J., Ajmo, C., Sanberg, C., Sanberg, P., Pennypacker, K., and Willing, A.: Cord blood treatment leads to full recovery in rats with experimental stroke. Cell Transplantation (in press), 2005.

Nikonenko I, Bancila M, Bloc A, Muller D and Bijlenga P (2005) Inhibition of T-type calcium channels protects neurons from delayed ischemia-induced damage. Mol. Pharmacol 68:84-89.

Nishikawa H, Hashino A, Kume T, Katsuki H, Kaneko S and Akaike A (2000) Involvement of direct inhibition of NMDA receptors in the effects of sigma-receptor ligands on glutamate neurotoxicity in vitro. Eur J Pharmacol 404:41-48.

Rawis S M, Baron D A, Geller E B and Adler M W (2002) Sigma sites mediate DTG-evoked hypothermia in rats. Pharmacol Biochem Behav 73 :779-786.

Robinson H P, Kawahara M, Jimbo Y, Torimitsu K, Kuroda Y and Kawana A (1993) Periodic synchronized bursting and intracellular calcium transients elicited by low magnesium in cultured cortical neurons. J Neurophysiol 70: 1606-1616.

Romieu P, Meunier J, Garcia D, Zozime N, Martin-Fardon R, Bowen W D and Maurice T (2004) The sigma I (sigma 1) receptor activation is a key step for the reactivation of cocaine conditioned place preference by drug priming. Psychopharmacology (Bed) 175:154-162.

Schurr A (2004) Neuroprotection against ischemic/hypoxic brain damage: blockers of ionotropic glutamate receptor and voltage sensitive calcium channels. Curr Drug Targets 5:603-618.

Senda T, Matsuno K, Okamoto K, Kobayashi T, Nakata K and Mita S (1996) Ameliorating effect of SA4503, a novel sigma I receptor agonist, on memory impairments induced by cholinergic dysfunction in rats. Eur J Pharmacol 315:1-10.

Siesjo, B. K., Katsura, K., and Kristian, T. (1996). Acidosis-related damage. Adv. Neurol. 71. 209-233.

Sircar R, Rappaport M, Nichtenhauser Rand Zukin S R (1987) The novel anticonvulsant MK801: a potent and specific ligand of the brain phencyclidine/sigma-receptor. Brain Res 435:235-240.

Skuza G and Rogoz Z (2003) Sigmal receptor antagonists attenuate antidepressant-like effect induced by co-administration of 1,3 di-o-tolylguanidine (DTG) and memantine in the forced swimming test in rats. PoIJ Pharmacol 55:1149-1152.

Soriani 0, Vaudry H, Mel Y A, Roman F and Cazin L (1998) Sigma ligands stimulate the electrical activity of frog pituitary melanotrope cells through a G-protein-dependent inhibition of potassium conductances. J Pharmacol Exp Ther 286:163-171.

Takahashi H, Kirsch J R, Hashimoto K, London E D, Koehler R C and Traystman R J (1996) PPBP [4-phenyl-1-(4-phenylbutyl)piperidine] decreases brain injury after transient focal ischemia in rats. Stroke 27:2120-2123.

Tanaka E, Yamamoto S, Kudo Y, Mihara S and Higashi H (1997) Mechanisms underlying the rapid depolarization produced by deprivation of oxygen and glucose in rat hippocampal CA1 neurons in vitro. J Neurophysiol 78:891-902.

Tang F, Dent E W and Kalil K (2003) Spontaneous calcium transients in developing cortical neurons regulate axon outgrowth. J Neurosci 23:927-936.

Thurgur C and Church J (1998) The anticonvulsant actions of sigma receptor ligands in the Mg2+-free model of epileptiform activity in rat hippocampal slices. Br J Pharmacol 124:917-929

Tombaugh, G. C., and Sapolsky, R. M. (1993). Evolving concepts about the role of acidosis in ischemic neuropathology. J. Neurochem. 61. 793-803.

Vendrame M, Gemma C, de Mesquita D, Collier L, Bickford P C, Sanberg C D, Sanberg P R, Pennypacker K R, and A E, W.: Anti-inflammatory effects of human cord blood cells in a rat model of stroke. Stem Cells and Development. in press, 2005.

Vendrame, M., Cassady, J., Newcomb, J., Butler, T., Pennypacker, K. R., Zigova, T., Sanberg, C. D., Sanberg, P. R., and Willing, A. E.: Infusion of human umbilical cord blood cells in a rat model of stroke dose-dependently rescues behavioral deficits and reduces infarct volume. Stroke 35 (10): 2390-5, 2004.

Vilner B J and Bowen W D (2000) Modulation of cellular calcium by sigma-2 receptors: release from intracellular stores in human SK-N-SH neuroblastoma cells. J Pharmacol Exp Ther 292:900-911.

Waldmann, R., Champigny, G., Bassilana, F., Heurteaux, C., and Lazdunski, M. (1997a). A proton-gated channel involved in acid-sensing. Nature. 386. 173-177.

Walker J M, Bowen W D, Walker F O, Matsumoto R R, De Costa B and Rice K C (1990) Sigma receptors: biology and function. Pharmacol Rev 42:355-402.

Wang Y and Lee H K (1991) Electrophysiological interactions between NMDA and phencyclidine/sigma receptor agonists and antagonists in Purkinje neurons in the cerebellum of the rat. Neuropharmacology 30:985-994.

Wemmie, J. A., Chen, J., Askwith, C. C., Hruska-Hageman, A. M., Price, M. P., Nolan, B. C., Yoder, P. G., Lamani, E., Hoshi, T. Freeman, J. H., and Welsh, M. J. (2002). The acid-activated ion channel ASIC contributes to synaptic plasticity, lerning and memory. Neuron. 34. 463-477.

Xiong, Z. G., Zhu, X. M., Chu, X. P., Minami, M., and Hey, J. (2004). Neuroprotection in Ischemia: blocking calcium-permeable acid-sensing ion channels. Cell. 118: 687-698.

Yermolaieva, O., Leonard, A. S., Schnizler, M. K., Abboud, F. M., and Welsh, M. J. (2004). Extracellular acidosis increases neuronal cell calcium by activating acid-sensing ion channel 1a. PNAS. 101. 6752-6757.

Zhang H and Cuevas J (2002) Sigma receptors inhibit high-voltage-activated calcium channels in rat sympathetic and parasympathetic neurons. J Neurophysiol 87:2867-2879.

Zhang H and Cuevas J (2005) sigma Receptor activation blocks potassium channels and depresses neuroexcitability in rat intracardiac neurons. J Pharmacol Exp Ther 313:1387-1396.

Zhang, H., Guerrero, W., DeMesquita, D., Pennypacker, K., Cuevas J. (2004) Sigma-1 receptor activation attenuates elevations in intracellular calcium evoked by chemical ischemia. Soc. Neurosci. Abst. 1019.1.

The disclosure of all publications cited above are expressly incorporated herein by reference, each in its entirety, to the same extent as if each were incorporated by reference individually.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A method of treating ischemic stroke comprising the step of administering a sigma receptor agonist to a patient in need thereof.

2. The method of claim 1 where the sigma receptor agonist is selected from the group consisting of 1,3-di-o-tolyguanidine (DTG), carbetapentane, (+)-pentazocine, PRE-084, rimcazole, L-687,384, BD-737, JO-1784(igmesine).

3. The method of claim 1 where the sigma receptor agonist is DTG.

4. The method of claim 1 wherein the sigma receptor agonist is a sigma-1 receptor agonist.

5. The method of claim 1 wherein the sigma receptor agonist is administered more than about three hours post-stroke.

6. The method of claim 1 wherein the sigma receptor agonist is administered about twenty four hours post-stroke.

7. A method of decreasing ischemia-induced elevations in intracellular calcium comprising the step of administering a sigma receptor agonist to a patient in need thereof.

8. The method of claim 7 where the sigma receptor agonist is selected from the group consisting of 1,3-di-o-tolyguanidine (DTG), carbetapentane, (+)-pentazocine, PRE-084, rimcazole, L-687,384, BD-737, JO-1784(igmesine).

9. The method of claim 7 where the sigma receptor agonist is DTG.

10. The method of claim 7 wherein the sigma receptor agonist is a sigma-1 receptor agonist.

11. The method of claim 7 wherein the sigma receptor agonist is administered more than about three hours post-stroke.

12. The method of claim 7 wherein the sigma receptor agonist is administered about twenty four hours post-stroke.
